(19) United States
(12) Patent Application Publication
(10) Pub. No.: US 2004/0192583 A1
(43) Pub. Date: Sep. 30, 2004

(54) Treatment of obesity and associated conditions with TGF-beta inhibitors

(76) Inventors: Satyanarayana Medicherla, Cupertino, CA (US); Andrew A. Protter, Palo Alto, CA (US); George F. Schreiner, Los Altos, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/742,689

(22) Filed: Dec. 18, 2003

Related U.S. Application Data
(60) Provisional application No. 60/435,856, filed on Dec. 19, 2002.

(51) Int. Cl. A61K 38/00

(52) U.S. Cl. 514/2

(57) ABSTRACT

The invention concerns the treatment obesity and associated conditions with TGF-β inhibitors. More specifically, the invention concerns the use of TGF-β inhibitors in the treatment of obesity, type 2 diabetes, and pathologic conditions associated with obesity or type 2 diabetes.

### CROSS-REFERENCE TO RELATED APPLICATIONS

This is a non-provisional application filed under 37 C.F.R. 1.53(b), claiming priority under 35 U.S.C. § 119(e) to Provisional Application Ser. No. 60/435,856, filed on Dec. 19, 2002.

### BACKGROUND OF THE INVENTION

1. Field of the Invention

The present invention concerns the treatment obesity and associated conditions with TGF-β inhibitors. More specifically, the invention concerns the use of TGF-β inhibitors in the treatment of obesity, type 2 diabetes, and pathologic conditions associated with obesity or type 2 diabetes.

2. Description of the Related Art

Obesity

Obesity, that develops when energy intake exceeds energy expenditure over time, is a major public health problem is most industrialized countries. Thus, obesity is a strong risk factor for the development of type 2 diabetes mellitus, a disease characterized by insulin resistance, relative insulin hyposecretion, and hyperglycemia. There is also a close link between obesity and the development of high blood pressure and cardiovascular disease. While the factors contributing to obesity are not well understood, numerous studies show significant involvement of genetic factors.

A protein hormone called leptin has been discovered to play a role in regulation of the energy balance (Zhang et al., Nature 372:425-432 (1994)). This 167-amino acid protein containing a 21 -amino acid signal sequence is produced and secreted by mature adipocytes. The level of circulating leptin has been reported to be directly proportional to the total amount of fat in the body. Absence of the protein in mutant ob/ob mice leads to extreme obesity and type 2 diabetes mellitus. The leptin receptor (OB-R) has been found in the choroid plexus and hypothalamus and produced by expression cloning (see, e.g. Tartaglia. et al., Cell 83:1265-1271 (1995); Tartaglia et al., J. Biol. Chem. 272:6093-6096 (1997)). Both leptin and its receptor are important targets of anti-obesity drug development, however, the research has so far not yielded an effective, commercially available drug product to treat obesity.

Transforming Growth Factor-Beta

Transforming growth factor-beta (TGF-β) denotes a family of proteins, TGF-β1, TGF-β2, and TGF-β3, which are pleiotropic modulators of cell growth and differentiation, embryonic and bone development, extracellular matrix formation, hematopoiesis, immune and inflammatory responses (Roberts and Spom Handbook of Experimental Pharmacology (1990) 95:419-58; Massague et al. Ann Rev Cell Biol (1990) 6:597-646). Other members of this superfamily include activin, inhibin, bone morphogenic protein, and Mullerian inhibiting substance. TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication.

TGF-β, including TGF-β1, -β2 and -β3, exerts its biological activities through a receptor system including the type I and type II single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGFβ-R1) by the type II receptor (TGFβ-R2). The activated TGFβ-R1 phosphorylates a receptor-associated co-transcription factor Smad2/Smad3, thereby releasing it into the cytoplasm, where it binds to Smad4. The Smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intracellular communication. Other signaling pathways like the MAP kinase-ERK cascade are also activated by TGF-β signaling. For review, see, e.g. Whitman, Genes Dev. 12:2445-62 (1998); and Miyazono et al., Adv. Immunol. 75:111-57 (2000), which are expressly incorporated herein by reference. Further information about the TGF-β signaling pathway can be found, for example, in the following publications: Attisano et al., “Signal transduction by the TGF-β superfamily” Science 296:1646-7 (2002); Bottinger and Bitzer, “TGF-β signaling in renal disease” Am. Soc. Nephrol. 13:2600-2610 (2002); Topper, J. N., “TGF-β in the cardiovascular system: molecular mechanisms of a context-specific growth factor” Trends Cardiovasc. Med. 10:132-7 (2000), review; Itoh et al., “Signaling of transforming growth factor-β family” Eur. J. Biochem. 267:6954-67 (2000), review.

### SUMMARY OF THE INVENTION

In one aspect, the invention concerns a method for the treatment of obesity or a pathologic condition associated with obesity comprising administering to an obese mammalian subject or a mammalian subject at risk of developing obesity a therapeutically effective amount of a compound capable of inhibiting TGF-β signaling through a TGF-β receptor.

In another aspect, the invention concerns a method for the treatment of type 2 diabetes comprising administering to a mammalian subject diagnosed with or at risk of developing type 2 diabetes a therapeutically effective amount of a compound capable of inhibiting TGF-β signaling through a TGF-β receptor.

In yet another aspect, the invention concerns a method for appetite suppression comprising administering to a mammalian subject in need an effective amount of a compound capable of inhibiting TGF-β signaling through a TGF-β receptor.

In a further aspect, the invention concerns a method for limiting food intake in a mammalian subject comprising administering to said subject an effective amount of a compound capable of inhibiting TGF-β signaling through a TGF-β receptor.

The invention further concerns pharmaceutical and dietary formulations for use in any of the foregoing methods, comprising a compound capable of inhibiting TGF-β signaling through a TGF-β receptor in admixture with at least one carrier.

In all embodiments, a preferred TGF-β receptor is a TGFβ-R1 kinase. In a particular embodiment, the compound capable of inhibiting TGF-β signaling through a TGF-β receptor binds to a TGFβ-R1 kinase. In another particular embodiment, the compound may additionally bind to at least one further receptor kinase, such as an activin receptor (Alk4).

The molecules used in practicing the present invention are preferably non-peptide small molecules, e.g. small organic molecules.

A preferred group of the small organic molecules of the present invention is represented by the formula (1):

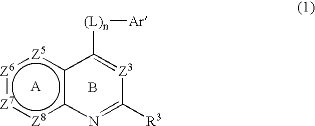

or the pharmaceutically acceptable salts or prodrug forms thereof; wherein:

R3 is a noninterfering substituent;

each Z is CR2 or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

Another group of the small organic molecules herein are represented by the formula (2)

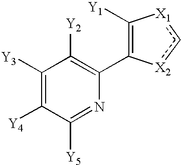

or the pharmaceutically acceptable salts or prodrug forms thereof; wherein:

Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ—Ph, —S—(CH₂)ₘ—Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S;

Y₂, Y₃, Y₄, and Y₅ independently represent hydrogen, alkyl(1-6C), alkoxy(1-6 C), haloalkyl(1-6 C), halo, NH₂, NH-alkyl(1-6C), or NH(CH₂)ₙ—Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙ—Ph, wherein n is 0-3, and the remainder of Y₂, Y₃, Y₄, and Y₅ represent hydrogen, alkyl(1-6 C), alkoxy(l-6C), haloalkyl(1-6 C), halo, NH2, NH-alkyl(1-6 C), or NH(CH₂)ₙ—Ph wherein n is 0-3; and

one of X₁ and X₂ is N and the other is NR₆, wherein R₆ is hydrogen or alkyl(1-6 C).

A further group of the small organic molecules herein is represented by the formula (3)

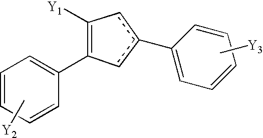

or the pharmaceutically acceptable salts or prodrug forms thereof; wherein:

Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)—Ph, —S—(CH₂)ₙ—Ph, cyano, phenyl, and CO₂R, wherein R is hydrogen or alkyl(1-6 C), and n is 0, 1, 2, or 3; or Y₁ represents phenyl fused with an aromatic or nonaromatic cyclic ring of 5-7 members wherein said cyclic ring optionally contains up to two heteroatoms, independently selected from N, O, and S;

Y₂ is H, NH(CH₂)ₙ—Ph or NH-alkyl(1-6 C), wherein n is 0, 1, 2, or 3;

Y₃ is CO₂H, CONH₂, CN, NO₂, alkylthio(1-6 C), —SO₂-alkyl(C1-6), alkoxy(C1-6), SONH₂, CONHOH, NH2, CHO, CH₂NH₂, or CO₂R, wherein R is hydrogen or alkyl(1-6 C);

one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

Yet another group of the small organic molecules herein is represented by the following formula (4)

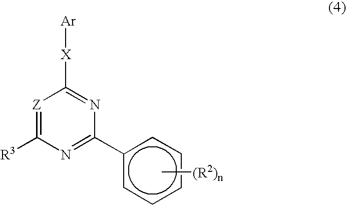

or the pharmaceutically acceptable salts or prodrug forms thereof; wherein:

Ar represents an optionally substituted aromatic or optionally substituted heteroaromatic moiety containing 5-12 ring members wherein said heteroaromatic moiety contains one or more O, S, and/or N with a proviso that the optionally substituted Ar is not

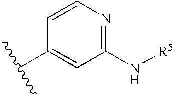

wherein R⁵ is H, alkyl (1-6C), alkenyl (2-6C), alkynyl (2-6C), an aromatic or heteroaromatic moiety containing 5-11 ring members;

X is NR′, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5.

In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

Further small organic compounds within the scope herein are represented by formula (5)

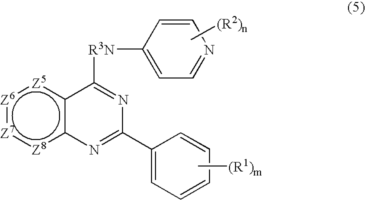

or the pharmaceutically acceptable salts or prodrug forms thereof; wherein:

each of Z⁵, Z⁶, Z⁷ and Z⁸ is N or CH and wherein one or two Z⁵, Z⁶, Z⁷ and Z⁸ are N and wherein two adjacent Z positions cannot be N;

m and n are each independently 0-3;

R¹ is halo, alkyl, alkoxy or alkyl halide and wherein two adjacent R¹ groups may be joined to form an aliphatic heterocyclic ring of 5-6 members;

R is a noninterfering substituent; and

R³ is H or CH₃.

### BRIEF DESCRIPTION OF THE DRAWINGS

FIG. 1 illustrates that db/db mice develop hyperphagia, obesity, hyperinsulemia, hyperleptinemia, hypertriglyceredmia, and hyperglycemia by 16 weeks of age and the complications of obesity by about 32 weeks.

FIG. 2 illustrates a study performed to evaluate the effect of TGF-β inhibitor on 16-week-old male diabetic db/db mice.

FIG. 3 illustrates plasma levels of a representative TGF-β inhibitor in db/db mice during the study.

FIG. 4 illustrates that the administration of 150 mg/kg/body weight/day of a representative TGF-β inhibitor significantly reduced the body weight of db/db obese mice.

FIG. 5 illustrates that the administration of a representative TGF-β inhibitor lowered the blood glucose level in lean and db/db obese mice.

FIG. 6 illustrates that the administration of 150 mg/kg/body weight/day of a representative TGF-β inhibitor significantly reduced the body weight of db/db obese mice.

FIG. 7 illustrates that the administration of a representative TGF-β inhibitor lowered the food intake of db/db mice in a statistically significant manner.

FIG. 8 illustrates that the administration of a representative TGF-β inhibitor reduced abdominal fat masses in db/db mice.

### DETAILED DESCRIPTION OF THE PREFERRED EMBODIMENT

### A. Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

The term “obesity” is used to describe an excessive amount of body fat. Typically, a person is considered obese if he or she has a body mass index (BMI) of 30 kg/M² or greater.

The term “pathologic condition associated with obesity” is used in the broadest sense and includes any condition that results, at least partially, from the long-term effects of obesity. Such conditions include, without limitation, type 2 diabetes, insulin resistance, sexual dysfunction, hypertension, hypercholesterolemia, atherosclerosis, hyperlipoproteinemia, and hypertriglyceridemia.

The terms “type 2 diabetes,” “type II diabetes,” type 2 diabetes mellitus,” “type II diabetes mellitus,” “non-insulin-dependent diabetes,” and “non-insulin-dependent diabetes mellitus (NIDDM)” are used interchangeably, and refer to a chronic diseases characterized by insulin resistance at the level of fat and muscle cells and resultant hyperglycemia.

The term “pathologic condition associated with type 2 diabetes” is used to refer to any condition that results, at least partially, from the long-term effects of type 2 diabetes. Such conditions include, without limitation, diabetic retinopathy, diabetic neuropathy, hypertension, atherosclerosis, diabetic ulcers, and in general damage caused to blood vessels, nerves and other internal structures by elevated blood sugar levels.

The term “TGF-β ” is used herein to include native sequence TGF-β1, TGF-β 2 and TGF-β 3 of all mammalian species, including any naturally occurring variants of the TGF-β polypeptides.

The term “biological activity mediated by a TGF-β receptor” and similar terms are used to refer to any activity associated with the activation of a TGF-β receptor, and downstream intracellular signaling events.

A “biological activity mediated by the TGFβ-R1 kinase receptor,” or “biological activity mediated by a TGFβ-R1 receptor” can be any activity associated with the activation of TGFβ-R1 and downsteam intracellular signaling events, such as the phosphorylation of Smad2/Smad3, or any signaling effect occurring in the Smad-independent signaling arm of the TGFβ signal transduction cascad, including, for example, p38 and ras.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Thus, in the case of obesity, the term “treatment” includes the treatment of obese subjects as well as preventative treatment of subjects a risk of developing obesity. Similarly, in the case of type 2 diabetes, “treatment” refers both to treating subjects diagnosed with type 2 diabetes and those at risk of developing type 2 diabetes.

The “pathology” of a disease or condition includes all phenomena that compromise the well-being of the patient.

The term “TGF-β inhibitor” as used herein refers to a molecule having the ability to inhibit a biological function of a native TGF-β molecule mediated by a TGF-β receptor kinase, such as the TGFβ-R1 or TGFβ-R2 receptor, by interacting with a TGF-β receptor kinase. Accordingly, the term “inhibitor” is defined in the context of the biological role of TGF-β and its receptors. While the inhibitors herein are characterized by their ability to interact with a TGF-β receptor kinase and thereby inhibiting TGF-β biological function, they might additionally interact with other members in the TGF-β signal transduction pathway or members shared by the TGF-β signal transduction pathway and another pathway. Thus, the term “TGF-β inhibitor” specifically includes molecules capable of interacting with and inhibiting the biological function of two or more receptor kinases, including, without limitation, an activin receptor kinase, e.g. Alk4, and/or a MAP kinase.

The term “interact” with reference to an inhibitor and a receptor includes binding of the inhibitor to the receptor as well as indirect interaction, which does not involve binding. The binding to a receptor can, for example, be specific or preferential.

The terms “specifically binding,” “binds specifically,” “specific binding,” and grammatical variants thereof, are used to refer to binding to a unique epitope within a target molecule, such as a TGFβ receptor, e.g. the type I TGF-β receptor (TGFβ-R1). The binding must occur with an affinity to effectively inhibit TGF-β signaling through the receptor, e.g. TGFβ-R1.

The terms “preferentially binding,” binds preferentially,” “preferential binding,” and grammatical variants thereof, as used herein means that binding to one target is significantly greater than binding to any other binding partner. The binding affinity to the preferentially bound target is generally at least about two-fold, more preferably at least about five-fold, even more preferably at least about ten-fold greater than the binding affinity to any other binding partner.

The term “preferentially inhibit,” as used herein means that the inhibitory effect on the target that is “preferentially inhibited” is significantly greater than on any other target. Thus, for example, in the context of preferential inhibition of TGF-β-R1 kinase relative to the p38 kinase, the term means that the inhibitor inhibits biological activities mediated by the TGF-β-R1 kinase significantly more than biological activities mediated by the p38 kinase. The difference in the degree of inhibition, in favor of the preferentially inhibited receptor, generally is at least about two-fold, more preferably at least about five-fold, even more preferably at least about ten-fold.

The term “mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

The term “therapeutically effective amount” means an amount of a compound or combination of compounds that ameliorates, attenuates, or eliminates one or more symptoms of a particular disease or condition or prevents or delays the onset of one of more symptoms of a particular disease or condition.

As used herein, a “noninterfering substituent” is a substituent which leaves the ability of a compound of the invention to inhibit TGF-β activity qualitatively intact. Thus, the substituent may alter the degree of inhibition. However, as long as the compound of the invention retains the ability to inhibit TGF-β activity, the substituent will be classified as “noninterfering.”

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when indicated, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, the terms “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one ore more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuiranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

The pyridyl moiety, may also comprise two substituents which, when together, form a 5-7 membered carbocyclic or heterocyclic aliphatic ring.

The compounds of the invention may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound, the compound may also be supplied as a salt with a pharmaceutically acceptable cation, or may be supplied as an ester or free base.

### B. Modes of Carrying Out the Invention Obesity and Related Pathologic Conditions

As discussed before, obesity is a risk factor for the development of a series of pathologic conditions, including atherosclerosis, hypertension, cardiovascular disease, and various metabolic disorders, such as hypertriglyceridemia, hyperinsulinemia, and non-insulin dependent diabetes mellitus (type 2 diabetes).

Obesity often precedes the development of metabolic disorders, like those listed above, especially if it is characterized by a significant increase in abdominal visceral fat. Thus, obesity is considered the main risk factor for the development of type 2 diabetes mellitus in both adults and children. Type 2 diabetes typically develops over a longer period of time, and often is preceded by a condition called prediabetes. This condition occurs when blood glucose levels are higher than healthy levels but too low to be diagnosed as diabetes. Without lifestyle changes or other treatment, most people who have been diagnosed with prediabetes will progress to type 2 diabetes within 10 years. Pathologic conditions related to obesity specifically include pathologic conditions associated with type 2 diabetes mellitus, such as diabetic retinopathy, diabetic neuropathy, high blood pressure, atherosclerosis, diabetic ulcers, and in general damage caused to blood vessels, nerves and other internal structures by elevated blood sugar levels.

Animal Models of Obesity and Type 2 diabetes

Obesity is most frequently studied in mouse models. In 1994, the ob gene, encoding the protein hormone leptin was identified in genetically obese mice. Mice with mutations in the ob gene (ob/ob mice) are unable to produce leptin, and develop severe obesity. When leptin is administered to these mice, the mice decrease their food intake, their metabolic rate increases, and they lose a significant amount of weight. At present, ob/ob mice represent perhaps the best-studied and most convenient animal model of obesity.

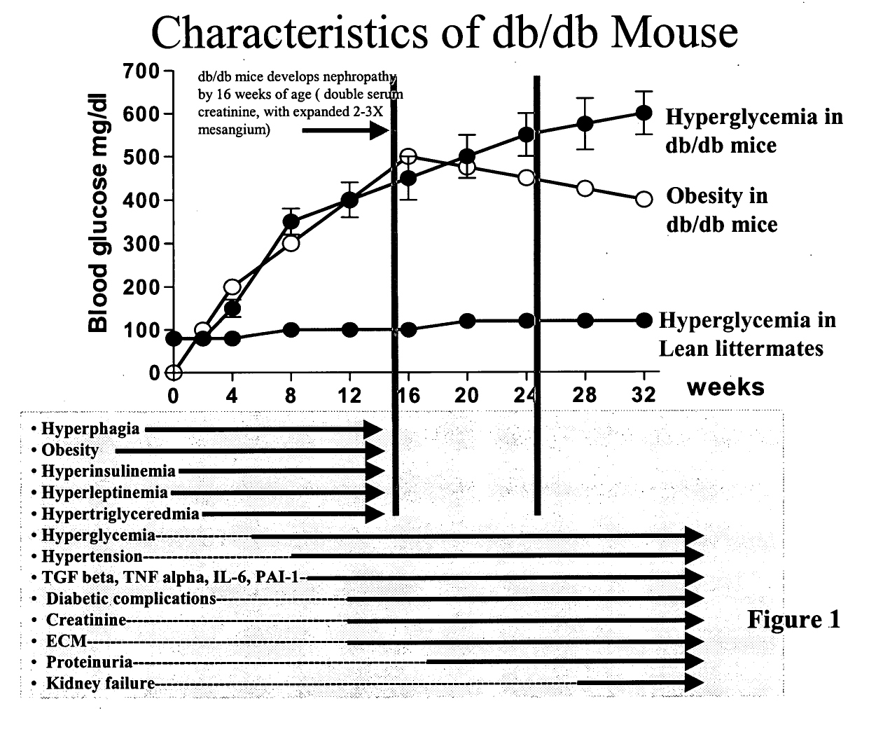

Another useful mouse model (db/db mice) is characterized by the presence of an abnormally spliced form of the ob receptor. Abnormal splicing results in the truncation of the cytoplasmic domain, which is essential for leptin signaling. As a result, db/db mice develop severe obesity and serve as an animal model of obesity and type 2 diabetes mellitus. More detailed characteristics of the db/db mice are illustrated in FIG. 1. As shown in FIG. 1, db/db mice develop hyperphagia, obesity, hyperinsulemia, hyperleptinemia, hypertriglyceredmia, and hyperglycemia by 16 weeks of age. By about 32 weeks, the mice develop complications of obesity, including hypertension, diabetic complications, excessive extracellular matrix (ECM) production, proteinuria, and in some instances kidney failure. In addition, the mice show elevated levels of creatinine, TGF-β, TNF-α, IL-6, and PAI-1.

Another gene that is apparently involved in the development of obesity is the fat gene (carboxypeptidase E), which is necessary for proteolytic processing of proinsulin and possibly other hormones. It has been found that mice containing mutations in the fat gene gradually develop obesity as they age, and show hyperglycemia that can be suppressed by the administration of insulin. Accordingly, fat/fat mice provide a further useful animal model for obesity studies.

Mutations in the tub gene encoding the insulin signaling protein TUB also yield a useful animal model of obesity. The “tubby” mutation introduces a splice site at the junction of the 3′ coding sequence that leads to loss of a 260 amino acid carboxy terminal domain, characteristic of the TUB family of proteins. The tub/tub mice exhibit hyperglycemia, increased levels of serum insulin, islet hypertrophy/hyperplasia, and beta-cell degranulation, and are useful models to study both obesity and diabetes.

The KK mouse is well known as a polygenic model for type 2 diabetes mellitus with moderate obesity (see, e.g. Suto et al., Mamm. Genome. 9:506-510 (1998).

The Zucker Diabetic Fatty (ZDF) rat is an inbred rat model that, through genetic mutations and a managed diet, mimics the characteristics of adult onset diabetes and related conditions. ZDF males homozygous for nonfunctional leptin receptors (fa/fa) develop obesity, hyperglycemia, and hyperlipidemia, and are widely used as an animal model of type 2 diabetes.

Obese SHR rats carrying the corpulent gene (cp), an allele of fatty (fa) develops characteristics associated with type 2 diabetes in humans (see, e.g. Michaelis and Hansen, ILAR News 32:19-22 (1990)).

The listed rodeni strains are commercially available.

In addition to genetic components, diet is a major factor in the development of obesity and type 2 diabetes. Just as in humans, an imbalance between energy intake and expenditure results in obesity in rodents. Accordingly, diet-induced rodent obesity models are convenient models of human obesity.

In one of such models, rats (e.g. Wistar rats) are made obese by feeding them “cafeteria diet.” This “cafeteria diet” consists of highly palatable and energy-dense foods for human consumption, such as cookies, Swiss cheese, salami, ham, crackers, etc., and has an energy content of approximately 10% protein, 30% carbohydrate and 60% fat (Zhou et al., J. Endocrinol. 159:165-172 (1998)). As a result of this diet, the rats develop obesity.

In other diet-induced rodent (rat or mouse) models, the animals are fed high fat/high carbohydrate diet and, as a result, develop obesity. For example, C57BLU6 male mice, available from The Jackson Laboratory (Bar Harbor, Me., USA), represent a commonly used mouse model for diet induced obesity (DIO).

End Points to Assess Efficacy of Anti-Obesity Treatment

The efficacy of a drug candidate to treat obesity and/or type 2 diabetes can be assessed in the foregoing and similar animal models, using a variety of end points. Suitable end points include, without limitation, monitoring the food intake, feeding behavior, body weight, locomotion, regional fat distribution, body compositions (carcass lipid and lean body fat-free mass), energy expenditure, glucose and insulin tolerance, serum and fat lipid profile, serum glucose and insulin profile and/or adipogenesis (in vivo or in vitro) of the experimental animals.

### C. Compounds of the invention

The compounds of the present invention are capable of inhibiting TGF-β signaling through a TGF-β receptor and find utility in the prevention and treatment of obesity and related pathologic conditions. A TGF-β inhibitor, as defined for the purpose of the present invention, can be any molecule having the ability to inhibit a biological function of a native TGF-β molecule mediated by a TGF-β receptor kinase, such as the TGFβ-R1 or TGFβ-R2 receptor via interaction with a TGF-β receptor kinase. Although the inhibitors are characterized by their ability to interact with a TGF-β receptor kinase and thereby inhibiting TGF-β biological function, they might additionally interact with other members in the TGF-β signal transduction pathway or members shared by the TGF-β signal transduction pathway and another pathway. Thus, TGF-β inhibitors might interact with two or more receptor kinases.

As discussed earlier, the type 1 and type 2 TGF-β receptors are serine-threonine kinases that signal through the Smad family of transcriptional regulators. Binding of TGF-β induces phosphorylation and activation of TGFβ-R1 by the TGF-R2. The activated TGFβ-R1 phosphorylates Smad2 and Smad3, which bind to Smad4 to move into the nucleus and form transcription regulatory complexes. Other signaling pathways, such as the MAP kinase-ERK cascade are also activated by TGF-β signaling, and modulate Smad activation. The Smad proteins couple the activation of both the TGF-β and the activin receptors to nuclear transcription. Thus, the TGF-β inhibitors of the present invention may additionally interact with an activin receptor kinase, such as Alk4, and/or a MAP kinase.

The compounds of the present invention include, without limitation, polypeptides, including antibodies and antibody-like molecules, peptides, polynucleotides, antisense molecules, decoys, and non-peptide small organic molecules that are capable of inhibiting TGF-β signaling through a TGF-β receptor.

In a particular embodiment, the compounds of the present invention are small organic molecules (non-peptide small molecules), generally less than about 1,000 daltons in size. Preferred non-peptide small molecules have molecular weights of less than about 750 daltons, more preferably less than about 500 daltons, and even more preferably less than about 300 daltons.

In a preferred embodiment, the compounds of the invention are of the formula (1):

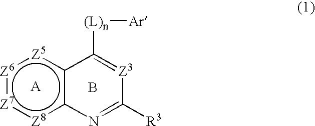

or the pharmaceutically acceptable salts or prodrug forms thereof; wherein:

R³ is a noninterfering substituent;

each Z is CR² or N, wherein no more than two Z positions in ring A are N, and wherein two adjacent Z positions in ring A cannot be N;

each R² is independently a noninterfering substituent;

L is a linker;

n is 0or 1; and

Ar′ is the residue of a cyclic aliphatic, cyclic heteroaliphatic, aromatic or heteroaromatic moiety optionally substituted with 1-3 noninterfering substituents.

In a preferred embodiment, the small organic molecules herein are derivatives of quinazoline and related compounds containing mandatory substituents at positions corresponding to the 2- and 4-positions of quinazoline. In general, a quinazoline nucleus is preferred, although alternatives within the scope of the invention are also illustrated below. Preferred embodiments for Z³ are N and CH; preferred embodiments for Z⁵-Z⁸ are CR². However, each of Z⁵-Z⁸ can also be N, with the proviso noted above. Thus, with respect to the basic quinazoline type ring system, preferred embodiments include quinazoline per se, and embodiments wherein all of Z⁵-Z⁸ as well as Z³ are either N or CH. Also preferred are those embodiments wherein Z³ is N, and either Z⁵ or Z⁸ or both Z⁵ and Z⁸ are N and Z⁶ and Z⁷ are CH or CR². Where R² is other than H, it is preferred that CR² occur at positions 6 and/or 7. Thus, by way of example, quinazoline derivatives within the scope of the invention include compounds comprising a quinazoline nucleus, having an aromatic ring attached in position 2 as a non-interfering substituent (R³), which may be further substituted.

With respect to the substituent at the positions corresponding to the 4-position of quinazoline, LAr′, L is present or absent and is a linker which spaces the substituent Ar′ from ring B at a distance of 2-8 Å, preferably 2-6 Å, more preferably 2-4 Å. The distance is measured from the ring carbon in ring B to which one valence of L is attached to the atom of the Ar′ cyclic moiety to which the other valence of the linker is attached. The Ar′ moiety may also be coupled directly to ring B (i.e., when n is 0). Typical, but nonlimiting, embodiments of L are of the formula S(CR²₂)ₘ, —NR¹SO₂(CR²₂)ₗ, NR¹(CR²₂)ₘ, NR¹CO(CR²₂)ₗ, O(CR²₂)ₘ, OCO(CR²₂)ₗ, and

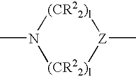

wherein Z is N or CH and wherein m is 0-4 and 1 is 0-3, preferably 1-3 and 1-2, respectively. L preferably provides —NR1- coupled directly to ring B. A preferred embodiment of R1 is H, but R1 may also be acyl, alkyl, arylacyl or arylalkyl where the aryl moiety may be substituted by 1-3 groups such as alkyl, alkenyl, alkynyl, acyl, aryl, alkylaryl, aroyl, N-aryl, NH-alkylaryl, NH-aroyl, halo, OR, NR₂, SR, —SOR, —NRSOR, —NRSO₂R, —SO₂R, —OCOR, —NRCOR, —NRCONR₂, —NRCOOR, —OCONR₂, —RCO, —COOR, —SO₃R, —CONR₂, SO₂NR₂, CN, CF₃, and NO₂, wherein each R is independently H or alkyl (1-4C), preferably the substituents are alkyl (1-6C), OR, SR or NR₂ wherein R is H or lower alkyl (1-4C). More preferably, R¹ is H or alkyl (1-6C). Any aryl groups contained in the substituents may further be substituted by for example alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, CN, CF₃, or NO₂, wherein each R is independently H or alkyl (1-4C).

Ar′ is aryl, heteroaryl, including 6-5 fused heteroaryl, cycloaliphatic or cycloheteroaliphatic. Preferably Ar′ is phenyl, 2-, 3- or 4-pyridyl, indolyl, 2- or 4-pyrimidyl, benzimidazolyl, indolyl, preferably each optionally substituted with a group selected from the group consisting of optionally substituted alkyl, alkenyl, alkynyl, aryl, N-aryl, NH-aroyl, halo, OR, NR₂, SR, —OOCR, —NROCR, RCO, —COOR, —CONR₂, SO₂NR₂, CN, CF₃, and NO₂, wherein each R is independently H or alkyl (1-4C).

Ar′ is more preferably indolyl, 6-pyrimidyl, 3- or 4-pyridyl, or optionally substituted phenyl.

For embodiments wherein Ar′ is optionally substituted phenyl, substituents include, without limitation, alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, —OCONR₂, RCO, -COOR, —SO₃R, —CONR₂, SO₂NR₂, CN, CF₃, and NO₂, wherein each R is independently H or alkyl (1-4C). Preferred substituents include halo, OR, SR, and NR₂ wherein R is H or methyl or ethyl. These substituents may occupy all five positions of the phenyl ring, preferably 1-2 positions, preferably one position. Embodiments of Ar′ include substituted of unsubstituted phenyl, 2-, 3-, or 4-pyridyl, 2-, 4- or 6-pyrimidyl, indolyl, isoquinolyl, quinolyl, benzimidazolyl, benzotriazolyl, benzothiazolyl, benzofuranyl, pyridyl, thienyl, furyl, pyrrolyl, thiazolyl, oxazolyl, imidazolyl, and morpholinyl. Particularly preferred as an embodiment of Ar′ is 3- or 4-pyridyl, especially 4-pyridyl in unsubstituted form.

Any of the aryl moieties, especially the phenyl moieties, may also comprise two substituents which, when taken together, form a 5-7 membered carbocyclic or heterocyclic aliphatic ring.

Thus, preferred embodiments of the substituents at the position of ring B corresponding to 4-position of the quinazoline include 2-(4-pyridyl)ethylamino; 4-pyridylamino; 3-pyridylamino; 2-pyridylamino; 4-indolylamino; 5-indolylamino; 3-methoxyanilinyl; 2-(2,5-difluorophenyl)ethylamino-, and the like.

R³ is generally a hydrocarbyl residue (1-20C) containing 0-5 heteroatoms selected from O, S and N. Preferably R³ is alkyl, aryl, arylalkyl, heteroalkyl, heteroaryl, or heteroarylalkyl, each unsubstituted or substituted with 1-3 substituents. The substituents are independently selected from a group that includes halo, OR, NR₂, SR, —SOR, —SO₂R, —OCOR, —NRCOR, —NRCONR₂, —NRCOOR, —OCONR₂, RCO, —COOR, —SO₃R, NRSOR, NRSO₂R, —CONR₂, SO₂NR₂, CN, CF₃, and NO₂, wherein each R is independently H or alkyl (1-4C) and with respect to any aryl or heteroaryl moiety, said group further including alkyl (1-6C) or alkenyl or alkynyl. Preferred embodiments of R³ (the substituent at position corresponding to the 2-position of the quinazoline) comprise a phenyl moiety optionally substituted with 1-2 substituents preferably halo, alkyl (1-6C), OR, NR₂, and SR wherein R is as defined above. Thus, preferred substituents at the 2-position of the quinazoline include phenyl, 2-halophenyl, e.g., 2-bromophenyl, 2-chlorophenyl, 2-fluorophenyl; 2-alkyl-phenyl, e.g., 2-methylphenyl, 2-ethylphenyl; 4-halophenyl, e.g., 4-bromophenyl, 4-chlorophenyl, 4-fluorophenyl; 5-halophenyl, e.g. 5-bromophenyl, 5-chlorophenyl, 5-fluorophenyl; 2,4- or 2,5-halophenyl, wherein the halo substituents at different positions may be identical or different, e.g. 2-fluoro-4-chlorophenyl; 2-bromo-4-chlorophenyl; 2-fluoro-5-chlorophenyl; 2-chloro-5-fluorophenyl, and the like. Other preferred embodiments of R³ comprise a cyclopentyl or cyclohexyl moiety.

As noted above, R² is a noninterfering substituent.

Each R² is also independently a hydrocarbyl residue (1-20C) containing 0-5 heteroatoms selected from O, S and N. Preferably, R² is independently H, alkyl, alkenyl, alkynyl, acyl or hetero-forms thereof or is aryl, arylalkyl, heteroalkyl, heteroaryl, or heteroarylalkyl, each unsubstituted or substituted with 1-3 substituents selected independently from the group consisting of alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C). The aryl or aroyl groups on said substituents may be further substituted by, for example, alky, alkenyl, alkynyl, halo, OR, NR₂, SR, —SOR, —SO₂R, -OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). More preferably the substituents on R² are selected from R⁴, halo, OR⁴, NR⁴₂, SR⁴, —OOCR⁴, —NROCR⁴, —COOR⁴, R⁴CO, —CONR⁴₂, —SO₂NR⁴₂, CN, CF₃, and NO₂, wherein each R⁴ is independently H, or optionally substituted alkyl (1-6C), or optionally substituted arylalkyl (7-12C) and wherein two R⁴ or two substituents on said alkyl or arylalkyl taken together may form a fused aliphatic ring of 5-7 members.

R² may also, itself, be selected from the group consisting of halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C).

More preferred substituents represented by R² are those as set forth with regard to the phenyl moieties contained in Ar′ or R³ as set forth above. Two adjacent CR² taken together may form a carbocyclic or heterocyclic fused aliphatic ring of 5-7 atoms. Preferred R² substituents are of the formula R⁴, —OR⁴, SR⁴ or R⁴NH—, especially R⁴NH—, wherein R⁴ is defined as above. Particularly preferred are instances wherein R⁴ is substituted arylalkyl. Specific representatives of the compounds of formula (1) are shown in Tables 1-3 below. All compounds listed in Table 1 have a quinazoline ring system (Z³ is N), where the A ring unsubstituted (Z⁵-Z⁸ represent CH). The substituents of the B ring are listed in Table 1.

TABLE 1

| Compound | | | |
|---|---|---|---|
| No. | L | Ar′ | R³ |
| 1 | NH | 4-pyridyl | 2-chlorophenyl |
| 2 | NH | 4-pyridyl | 2,6-dichlorophenyl |
| 3 | NH | 4-pyridyl | 2-methylphenyl |
| 4 | NH | 4-pyridyl | 2-bromophenyl |
| 5 | NH | 4-pyridyl | 2-fluorophenyl |
| 6 | NH | 4-pyridyl | 2,6-difluorophenyl |
| 7 | NH | 4-pyridyl | Phenyl |
| 8 | NH | 4-pyridyl | 4-fluorophenyl |
| 9 | NH | 4-pyridyl | 4-methoxyphenyl |
| 10 | NH | 4-pyridyl | 3-fluorophenyl |
| 11* | N* | 4-pyridyl | Phenyl |
| ^{∂}12^{†} | N^{†} | 4-pyridyl | Phenyl |
| 13 | NHCH₂ | 4-pyridyl | Phenyl |
| 14 | NHCH₂ | 4-pyridyl | 4-chlorophenyl |
| 15 | NH | 3-pyridyl | Phenyl |
| 16 | NHCH₂ | 2-pyridyl | Phenyl |
| 17 | NHCH₂ | 3-pyridyl | Phenyl |
| 18 | NHCH₂ | 2-pyridyl | Phenyl |
| 19 | NHCH₂CH₂ | 2-pyridyl | Phenyl |
| 20 | NH | 6-pyrimidinyl | Phenyl |
| 21 | NH | 2-pyrimidinyl | Phenyl |
| 22 | NH | Phenyl | Phenyl |
| 23 | NHCH₂ | henyl | 3-chlorophenyl |
| 24 | NH | 3-hydroxyphenyl | Phenyl |
| 25 | NH | 2-hydroxyphenyl | Phenyl |
| 26 | NH | 4-hydroxyphenyl | Phenyl |
| 27 | NH | 4-indolyl | Phenyl |
| 28 | NH | 5-indolyl | Phenyl |
| 29 | NH | 4-methoxyphenyl | Phenyl |
| 30 | NH | 3-methoxyphenyl | Phenyl |
| 31 | NH | 2-methoxyphenyl | Phenyl |
| 32 | NH | 4-(2-hydroxyethyl)phenyl | Phenyl |
| 33 | NH | 3-cyanophenyl | Phenyl |
| 34 | NHCH₂ | 2,5-difluorophenyl | Phenyl |
| 35 | NH | 4-(2-butyl)phenyl | Phenyl |
| 36 | NHCH₂ | 4-dimethylaminophenyl | Phenyl |
| 37 | NH | 4-pyridyl | Cyclopentyl |
| 38 | NH | 4-pyridyl | Phenyl |
| 39 | NHCH₂ | 3-pyridyl | Phenyl |
| 40 | NH | 4-pyrimidyl | Phenyl |
| 41^{‡} | N | 4-pyridyl | Phenyl |
| 42 | NH | p-aminomethylphenyl | Phenyl |
| 43 | NHCH₂ | 4-aminophenyl | Phenyl |
| 44 | NH | 4-pyridyl | 3-chlorophenyl |
| 45 | NH | phenyl | 4-pyridyl |
| 46 | NH | 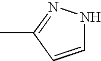 | Phenyl |
| 47 | NH | 4-pyridyl | t-butyl |
| 48 | NH | 2-benzylamino-3-pyridyl | Phenyl |
| 49 | NH | 2-benzylamino-4-pyridyl | Phenyl |
| 50 | NH | 3-benzyloxyphenyl | Phenyl |
| 51 | NH | 4-pyridyl | 3-aminophenyl |
| 52 | NH | 4-pyridyl | 4-pyridyl |
| 53 | NH | 4-pyridyl | 2-naphthyl |
| 54 | 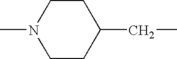 | 4-pyridyl | Phenyl |
| 55 | 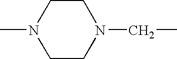 | phenyl | Phenyl |
| 56 | 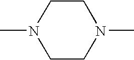 | 2-pyridyl | Phenyl |
| 57 | NHCH₂CH₂ | 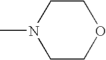 | Phenyl |
| 58 | not present | 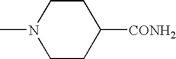 | Phenyl |
| 59 | not present | 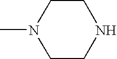 | Phenyl |
| 60 | NH | 4-pyridyl | Cyclopropyl |
| 61 | NH | 4-pyridyl | 2-trifluoromethyl |
| | | | phenyl |
| 62 | NH | 4-aminophenyl | Phenyl |
| 63 | NH | 4-pyridyl | Cyclohexyl |
| 64 | NH | 3-methoxyphenyl | 2-fluorophenyl |
| 65 | NH | 4-methoxyphenyl | 2-fluorophenyl |
| 66 | NH | 4-pyrimidinyl | 2-fluorophenyl |
| 67 | NH | 3-amino-4-pyridyl | Phenyl |
| 68 | NH | 4-pyridyl | 2-benzylaminophenyl |
| 69 | NH | 2-benzylaminophenyl | Phenyl |
| 70 | NH | 2-benzylaminophenyl | 4-cyanophenyl |
| 71 | NH | 3′-cyano-2- | Phenyl |
| | | benzylaminophenyl | |

*R¹ = 2-propyl
^{†}R¹ = 4-methoxyphenyl
^{‡}R¹ = 4-methoxyphenyl

The compounds in Table 2 contain modifications of the quinazoline shown. All of the compounds in Table 2 are embodiments of formula (1) wherein Z⁶ and Z⁷ represent CH. In all cases the linker, L, is present and is NH.

TABLE 2

| Compound No. | Z⁵ | Z⁸ | Ar′ | R³ |
|---|---|---|---|---|
| 72 | CH | N | 4-pyridyl | 2-fluorophenyl |
| 73 | CH | N | 4-pyridyl | 2-chlorophenyl |
| 74 | CH | N | 4-pyridyl | 5-chloro-2- |
| | | | | fluorphenyl |
| 75 | CH | N | 4-(3-methyl)-pyridyl | 5-chloro-2- |
| | | | | fluorphenyl |
| 76 | CH | N | 4-pyridyl | Phenyl |
| 77 | N | N | 4-pyridyl | phenyl |
| 78 | N | CH | 4-pyridyl | Phenyl |
| 79 | N | N | 4-pyridyl | 5-chloro-2- |
| | | | | fluorphenyl |
| 80 | N | N | 4-(3-methyl)-pyridyl | 5-chloro-2- |
| | | | | fluorphenyl |
| 81 | N | N | 4-pyridyl | 2-chlorophenyl |

Additional compounds were prepared wherein ring A contains CR² at Z⁶ or Z⁷ where R² is not H. These compounds, which are all quinazoline derivatives, wherein L is NH and Ar′ is 4-pyridyl, are shown in Table 3.

TABLE 3

| Compound | | |
|---|---|---|
| No. | R³ | CR² as noted |
| 82 | 2-chlorophenyl | 6,7-dimethoxy |
| 83 | 2-fluorophenyl | 6-nitro |
| 84 | 2-fluorophenyl | 6-amino |
| 85 | 2-fluorophenyl | 7-amino |
| 86 | 2-fluorophenyl | 6-(3-methoxybenzylamino) |
| 87 | 2-fluorophenyl | 6-(4-methoxybenzylamino) |
| 88 | 2-fluorophenyl | 6-(2-isobutylamino) |
| 89 | 2-fluorophenyl | 6-(4- |
| | | methylmercaptobenzylamino) |
| 90 | 2-fluorophenyl | 6-(4-methoxybenzoyl amino) |
| 91 | 4-fluorophenyl | 7-amino |
| 92 | 4-fluorophenyl | 7-(3-methoxybenzylamino) |

Although this embodiment of the invention is illustrated with reference to certain quinazoline derivatives, it is not so limited. Inhibitors of the present invention include compounds having a non-quinazoline, such as, a pyridine, pyrimidine nucleus carrying substituents like those discussed above with respect to the quinazoline derivatives.

The compounds of the invention, including compounds of the formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Another group of compounds for use in the methods of the present invention is represented by the following formula (2):

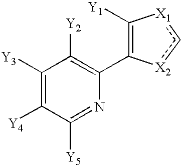

or the pharmaceutically acceptable salts or prodrug forms thereof; wherein:

Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ—Ph, —S—(CH₂)ₘ—Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S:

Y₂, Y₃, Y₄, and Y₅ independently represent hydrogen, alkyl(1-6C), alkoxy(1-6 C), haloalkyl(1-6 C), halo, NH₂, NH-alkyl(1-6C), or NH(CH₂)ₙ—Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙ—Ph, wherein n is 0-3, and the remainder of Y₂, Y₃, Y₄, and Y₅ represent hydrogen, alkyl(1-6 C), alkoxy(1-6C), haloalkyl(1-6 C), halo, NH2, NH-alkyl(1-6 C), or NH(CH₂)ₙ—Ph wherein n is 0-3; and

one of X₁ and X₂ is N and the other is NR₆, wherein R₆ is hydrogen or alkyl(1-6 C).

As used in formula (2), the double bonds indicated by the dotted lined represent possible tautomeric ring forms of the compounds. Further information about compounds of formula (2) and their preparation is disclosed in WO 02/40468, published May 23, 2002, the entire disclosure of which is hereby expressly incorporated by reference.

Yet another group of compounds for use in the methods of the invention is represented by the following formula (3):

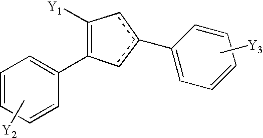

or the pharmaceutically acceptable salts or prodrug forms thereof; wherein:

Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)—Ph, —S—(CH₂)ₙ—Ph, cyano, phenyl, and CO₂R, wherein R is hydrogen or alkyl(l-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;

Y₂ is H, NH(CH₂)ₙ—Ph or NH-alkyl(l-6 C), wherein n is 0, 1, 2, or 3;

Y₃ is CO₂H, CONH₂, CN, NO₂, alkylthio(1-6 C), —SO₂—alkyl(C1-6), alkoxy(C1-6), SONH₂, CONHOH, NH₂, CHO, CH₂NH₂, or CO₂R, wherein R is hydrogen or alkyl(1-6 C);

one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

Further details of the compounds of formula (3) and their modes of preparation are disclosed in WO 00/61576 published Oct. 19, 2000, the entire disclosure of which is hereby expressly incorporated by reference.

In a further embodiment, the TGF-β inhibitors of the present invention are represented by the following formula (4):

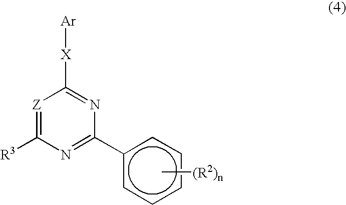

or the pharmaceutically acceptable salts or prodrug forms thereof; wherein:

Ar represents an optionally substituted aromatic or optionally substituted heteroaromatic moiety containing 5-12 ring members wherein said heteroaromatic moiety contains one or more O, S, and/or N with a proviso that the optionally substituted Ar is not

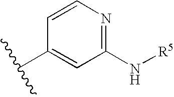

wherein R5 is H, alkyl (1-6C), alkenyl (2-6C), alkynyl (2-6C), an aromatic or heteroaromatic moiety containing 5-11 ring members;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or

R1 is H, alkyl (1-8C), alkenyl (2-8C), or alkynyl (2-8C); or

Z represents N or CR4; wherein

R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of said alkyl, each of which may optionally be substituted. Preferably R⁴ is H, alkyl (1-10C), OR, SR or NR₂ wherein R is H or alkyl (1-10C) or is O-aryl; or

R³ is defined in the same manner as R⁴ and preferred forms are similar, but R³ is independently embodied; or

each R² is independently alkyl (1-8C), alkenyl (2-8C), alkynyl (2-8C), acyl (1-8C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1-4C). Preferably R² is halo, alkyl (1-6C), OR, SR or NR₂ wherein R is H or lower alkyl (1-4C), more preferably halo; or n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or NO₂, wherein each R is independently H or lower alkyl (1-4C). Preferred substituents include alkyl, OR, NR₂, O-alkylaryl and NH-alkylaryl.

In general, any alkyl, alkenyl, alkynyl, acyl, or aryl group contained in a substituent may itself optionally be substituted by additional substituents. The nature of these substituents is similar to those recited with regard to the primary substituents themselves.

Representative compounds of formula (4) are listed in the following Table 4.

TABLE 4

| COMPOUND | |
|---|---|
| # | STRUCTURE |
| 93 | 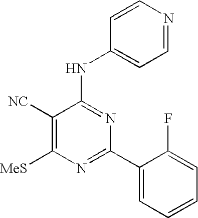 |
| 94 | 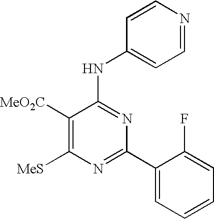 |
| 95 | 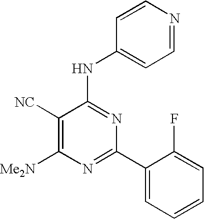 |
| 96 | 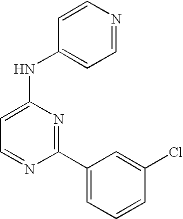 |
| 97 | 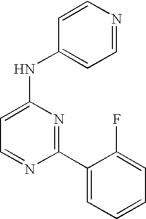 |
| 98 | 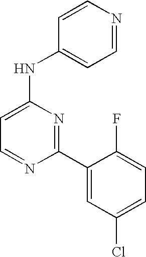 |
| 99 | 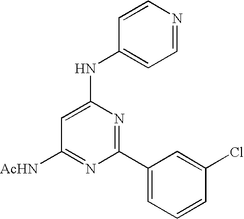 |
| 100 | 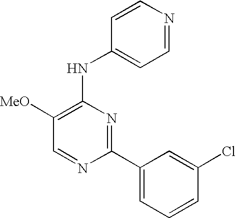 |
| 101 | 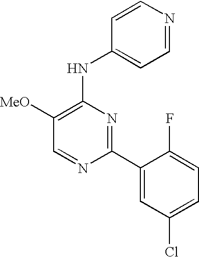 |
| 102 | 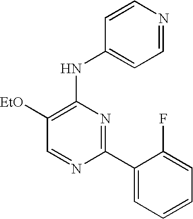 |
| 103 | 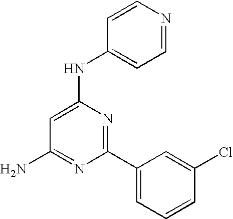 |
| 104 | 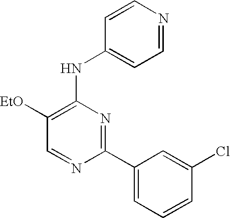 |
| 105 | 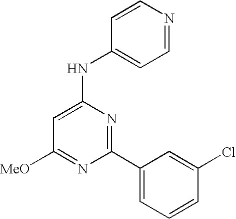 |
| 106 | 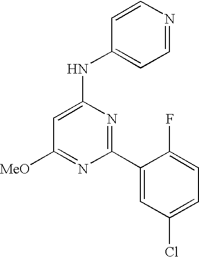 |
| 107 | 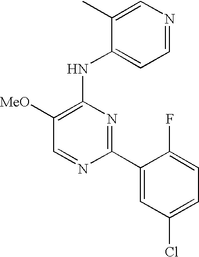 |
| 108 | 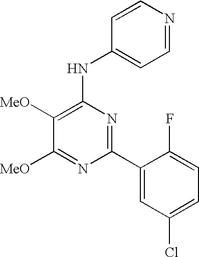 |
| 109 | 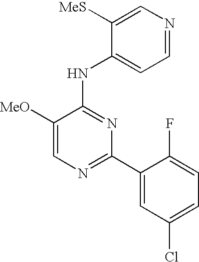 |
| 110 | 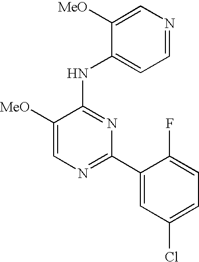 |
| 111 | 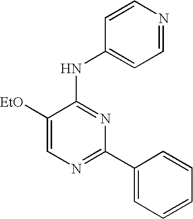 |
| 112 | 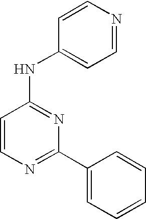 |
| 113 | 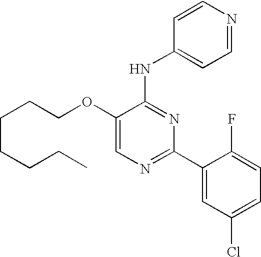 |
| 114 | 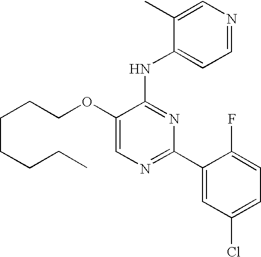 |
| 115 | 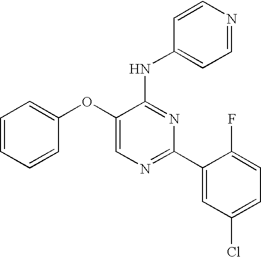 |
| 116 | 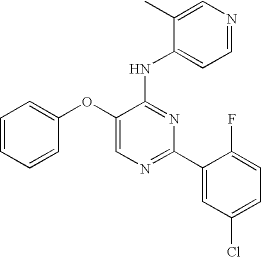 |
| 117 | 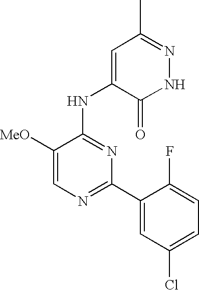 |
| 118 | 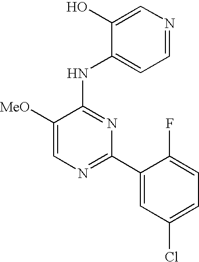 |
| 119 | 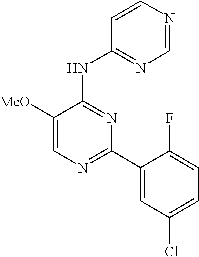 |
| 120 | 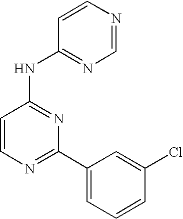 |
| 121 | 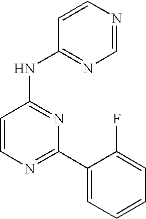 |
| 122 | 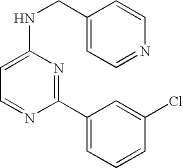 |
| 123 | 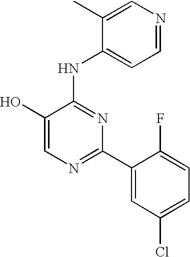 |
| 124 | 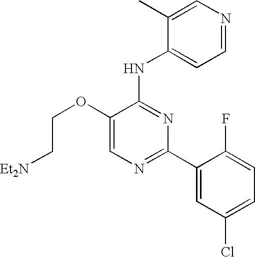 |
| 125 | 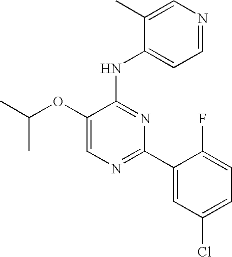 |
| 126 | 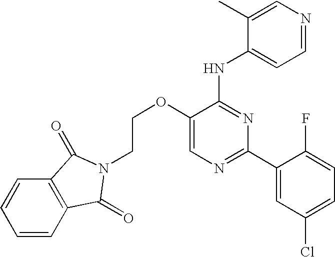 |
| 127 | 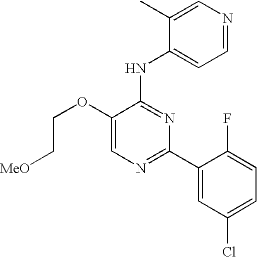 |
| 128 | 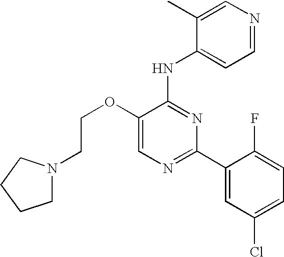 |
| 129 | 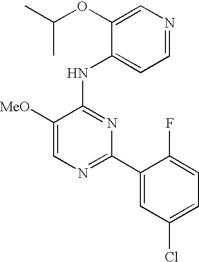 |
| 130 | 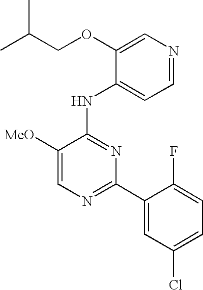 |
| 131 | 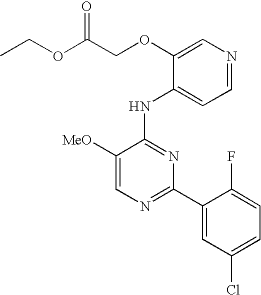 |
| 132 | 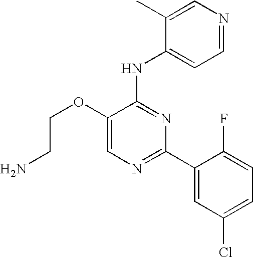 |
| 133 | 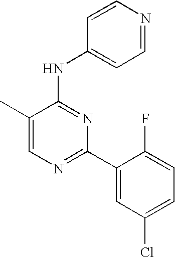 |
| 134 | 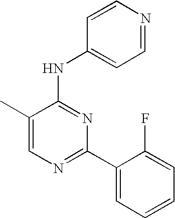 |
| 135 | 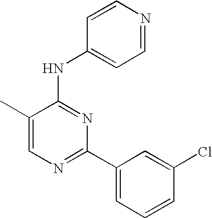 |
| 136 | 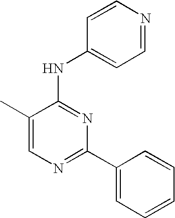 |
| 137 | 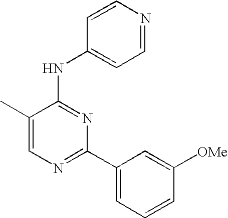 |
| 138 | 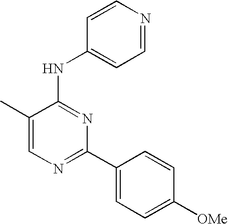 |
| 139 | 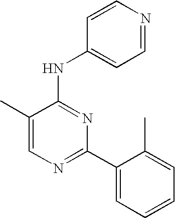 |
| 140 | 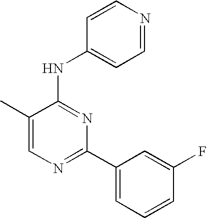 |
| 141 | 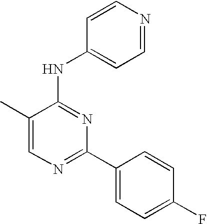 |
| 142 | 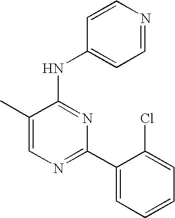 |
| 143 | 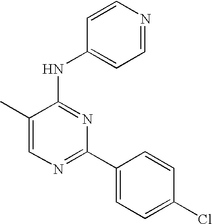 |
| 144 | 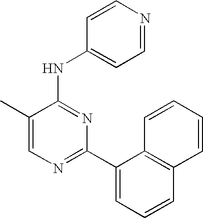 |
| 145 | 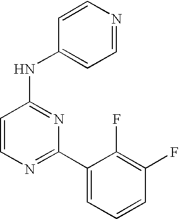 |
| 146 | 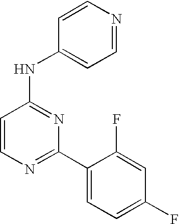 |
| 147 | 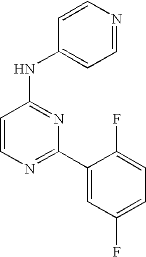 |
| 148 | 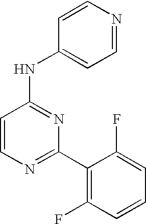 |
| 149 | 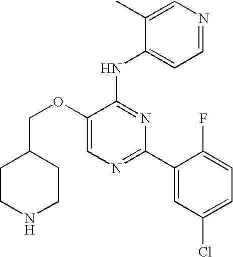 |
| 150 | 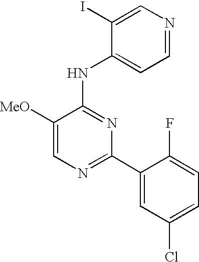 |
| 151 | 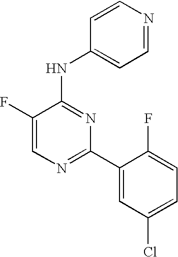 |
| 152 | 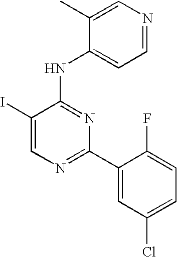 |
| 153 | 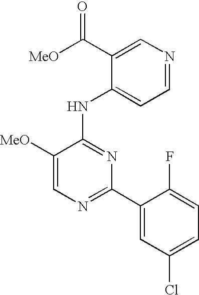 |
| 154 | 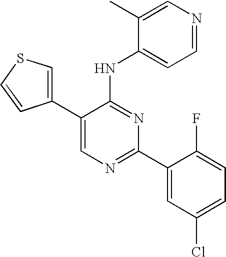 |
| 155 | 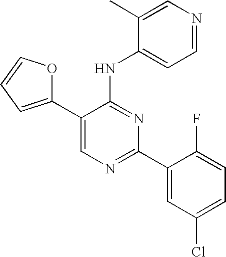 |
| 156 | 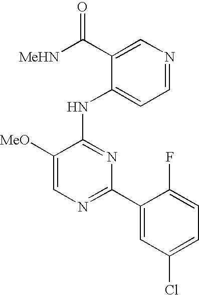 |
| 157 | 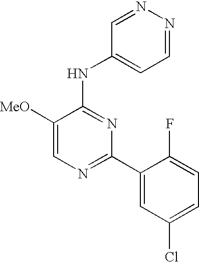 |
| 158 | 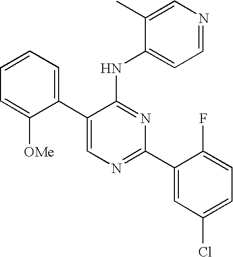 |
| 159 | 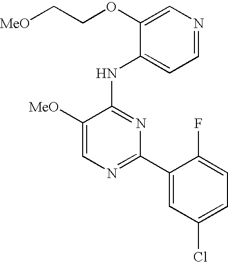 |
| 160 | 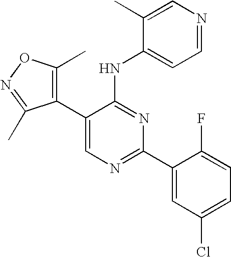 |
| 161 | 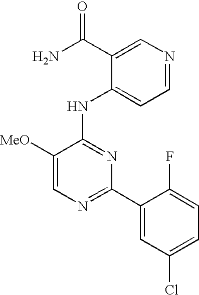 |
| 162 | 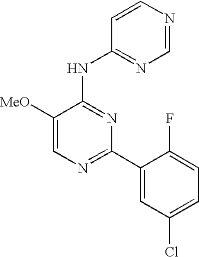 |
| 163 | 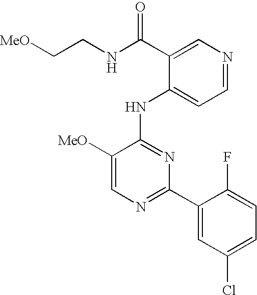 |
| 164 | 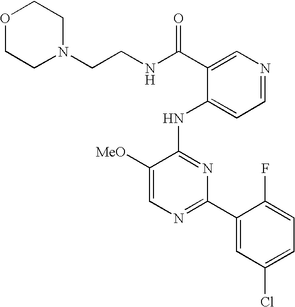 |
| 165 | 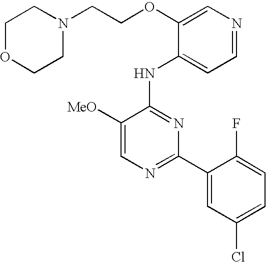 |
| 166 | 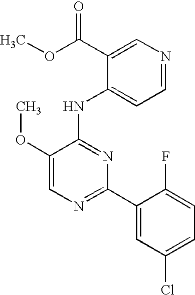 |
| 167 | 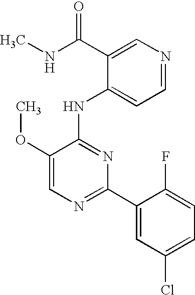 |
| 168 | 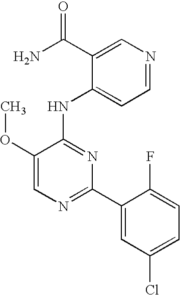 |
| 169 | 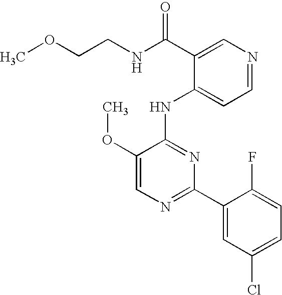 |
| 170 | 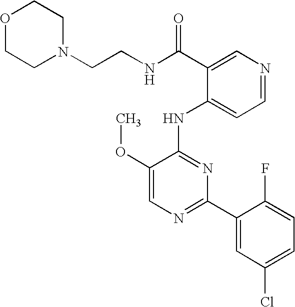 |
| 171 | 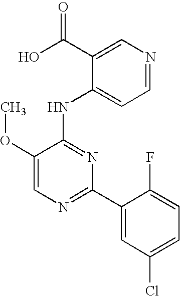 |
| 172 | 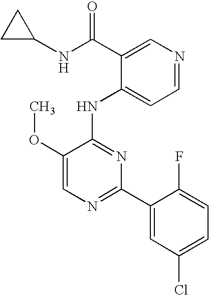 |
| 173 | 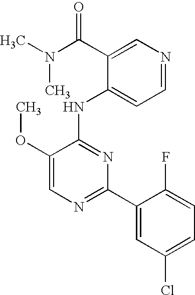 |
| 174 | 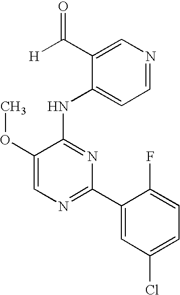 |
| 175 | 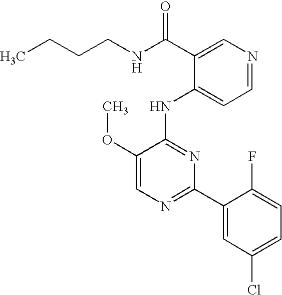 |
| 176 | 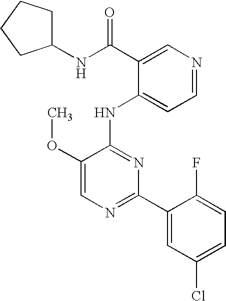 |
| 177 | 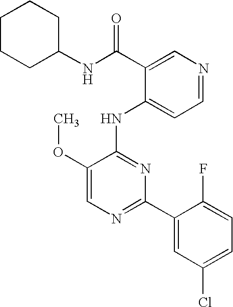 |
| 178 | 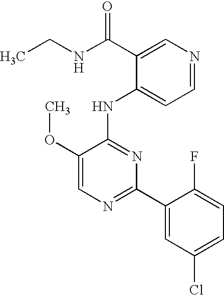 |
| 179 | 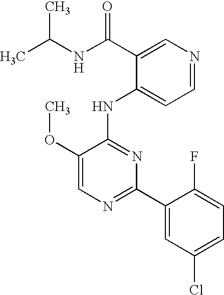 |
| 180 | 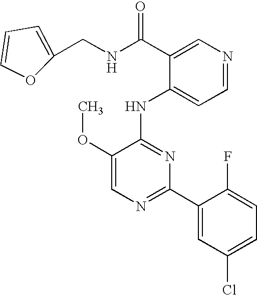 |
| 181 | 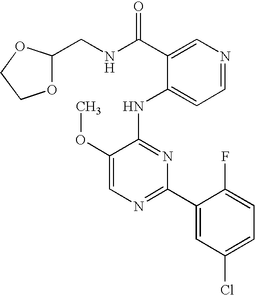 |
| 182 | 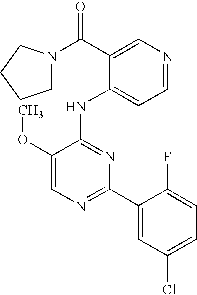 |
| 183 | 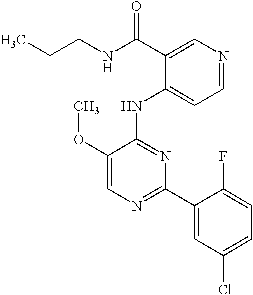 |
| 184 | 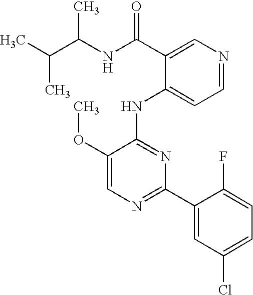 |
| 185 | 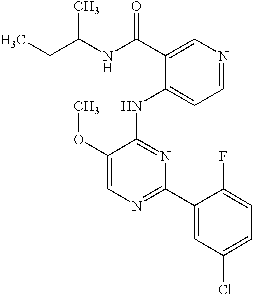 |
| 186 | 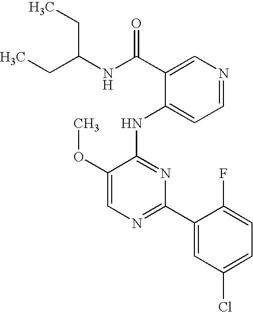 |
| 187 | 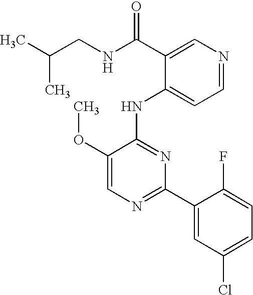 |
| 188 | 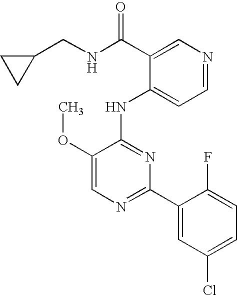 |
| 189 | 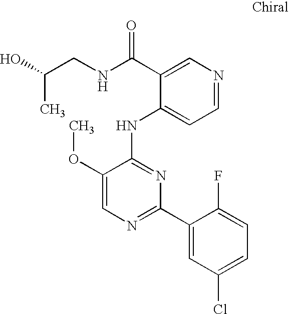 |
| 190 | 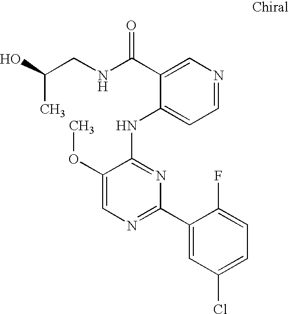 |
| 191 | 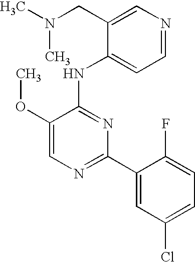 |
| 192 | 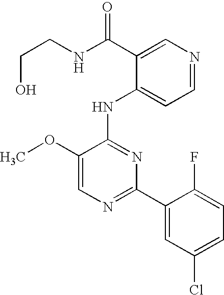 |
| 193 | 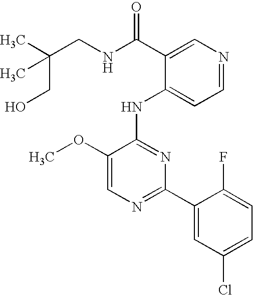 |
| 194 | 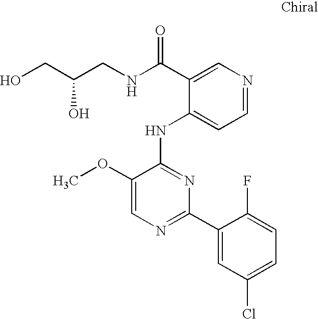 |
| 195 | 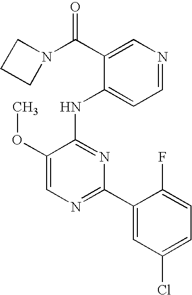 |
| 196 | 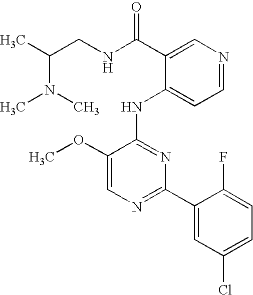 |
| 197 | 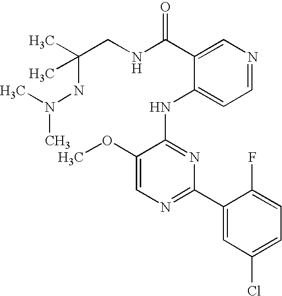 |
| 198 | 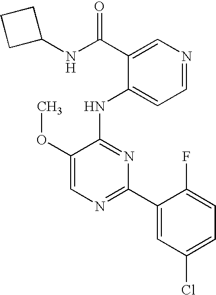 |
| 199 | 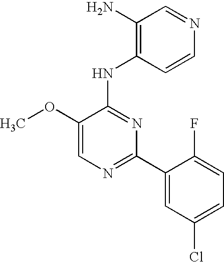 |
| 200 | 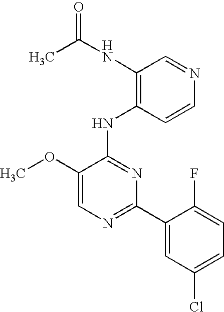 |
| 201 | 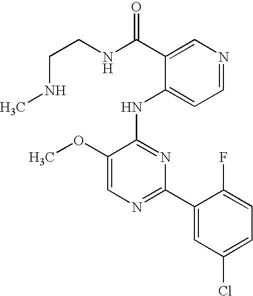 |
| 202 | 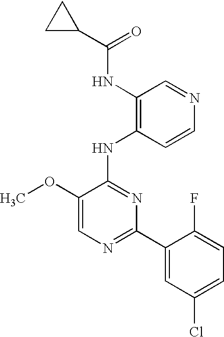 |
| 203 | 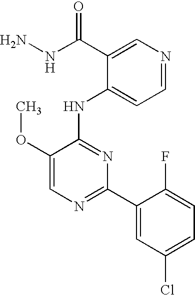 |
| 204 | 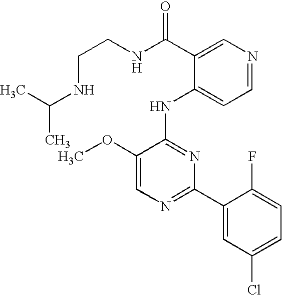 |
| 205 | 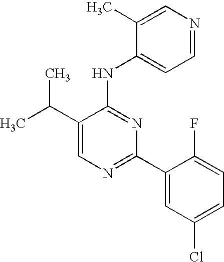 |
| 206 | 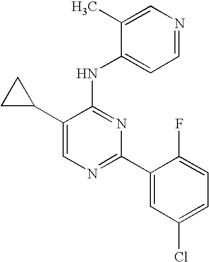 |
| 207 | 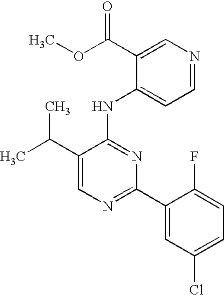 |
| 208 | 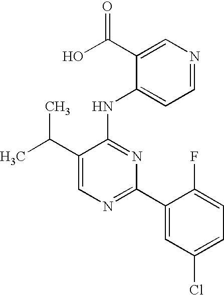 |
| 209 | 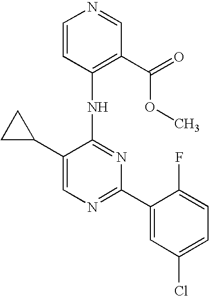 |
| 210 | 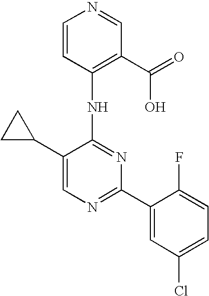 |
| 211 | 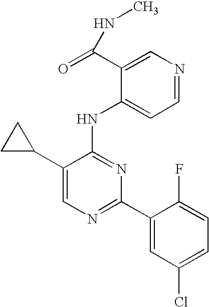 |
| 212 | 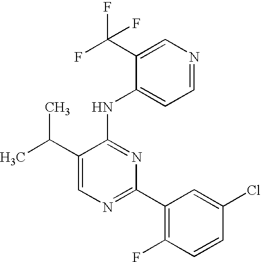 |
| 213 | 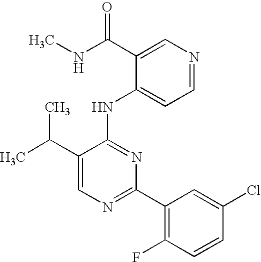 |
| 214 | 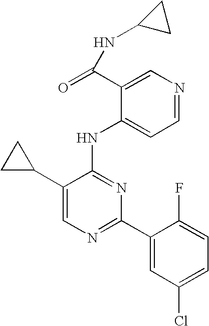 |
| 215 | 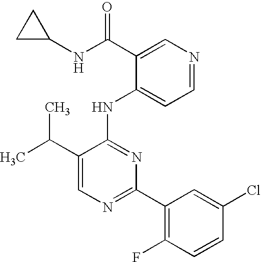 |
| 216 | 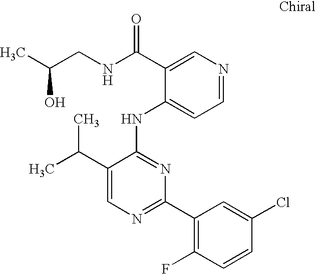 |
| 217 | 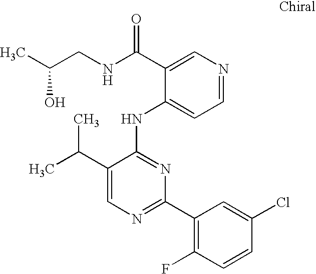 |
| 218 | 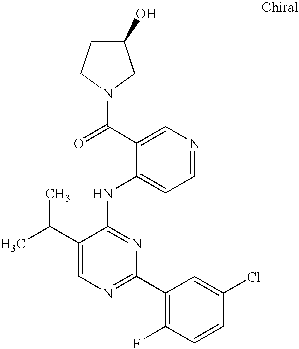 |
| 219 | 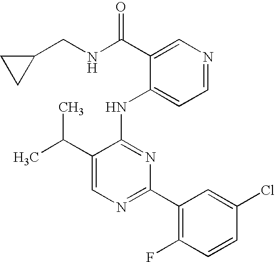 |
| 220 | 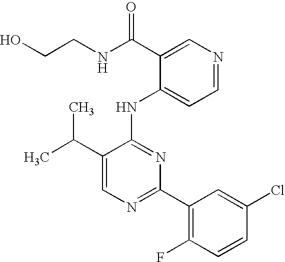 |
| 221 | 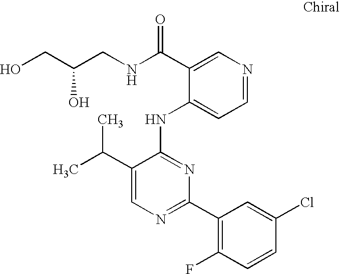 |
| 222 | 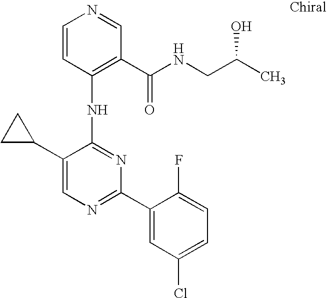 |
| 223 | 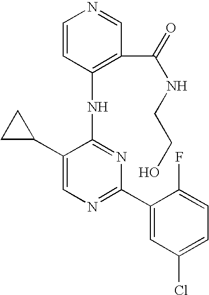 |
| 224 | 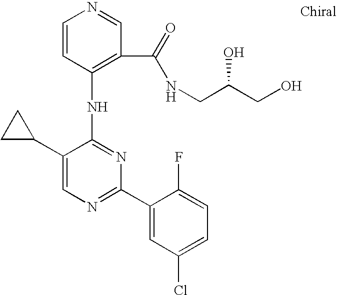 |
| 225 | 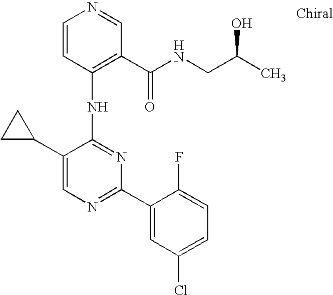 |
| 226 | 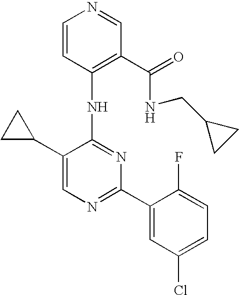 |
| 227 | 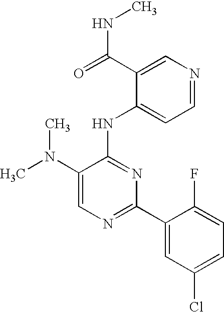 |
| 228 | 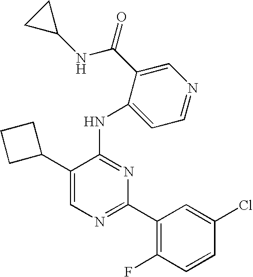 |
| 229 | 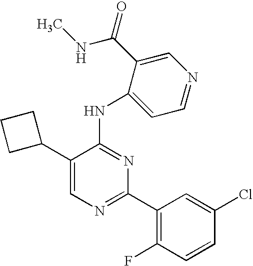 |
| 230 | 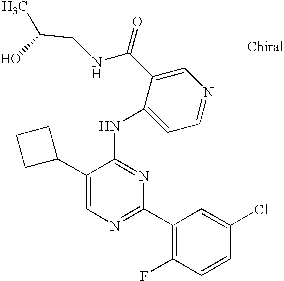 |
| 231 | 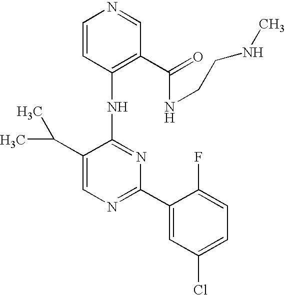 |
| 232 | 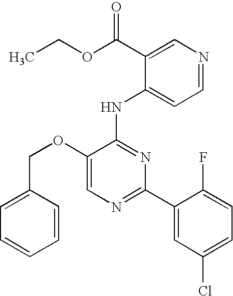 |
| 233 | 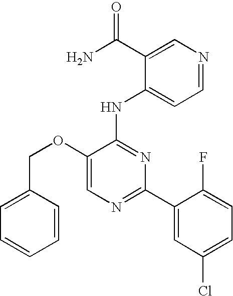 |
| 234 | 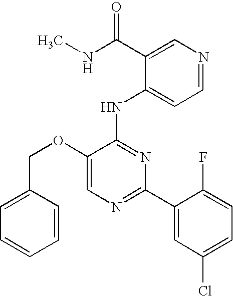 |
| 235 | 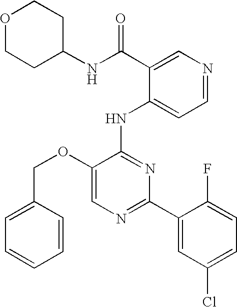 |
| 236 | 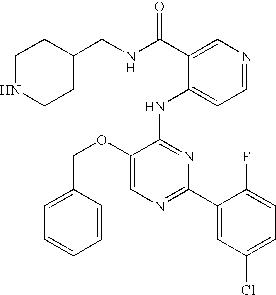 |
| 237 | 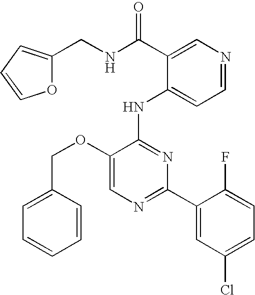 |
| 238 | 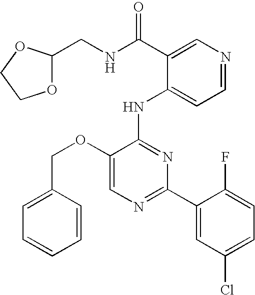 |
| 239 | 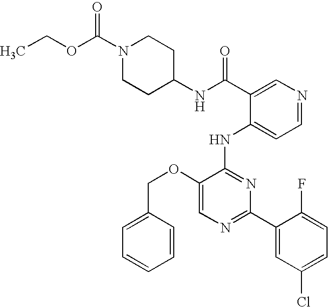 |
| 240 | 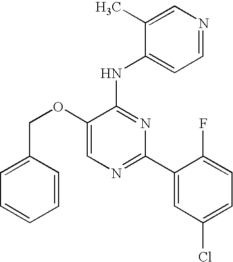 |
| 241 | 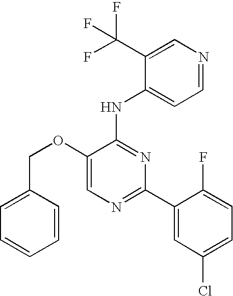 |
| 242 | 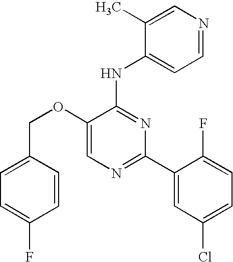 |
| 243 | 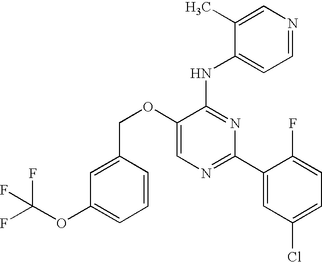 |
| 244 | 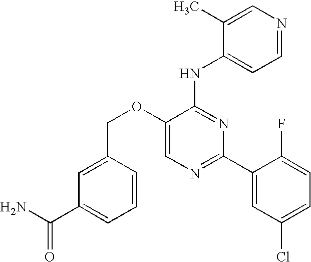 |
| 245 | 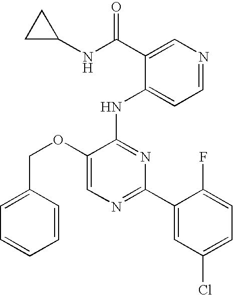 |
| 246 | 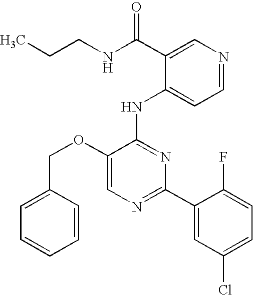 |
| 247 | 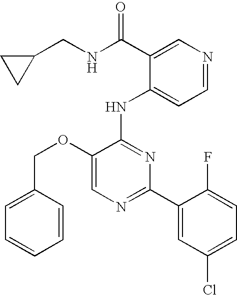 |
| 248 | 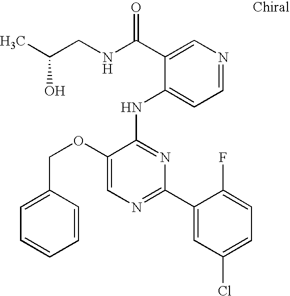 |
| 249 | 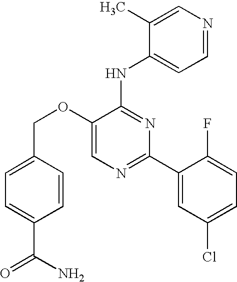 |
| 250 | 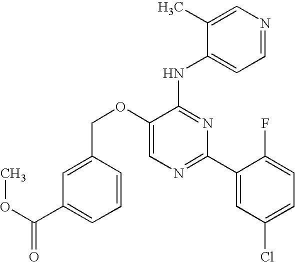 |
| 251 | 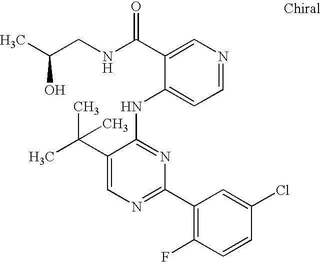 |
| 252 | 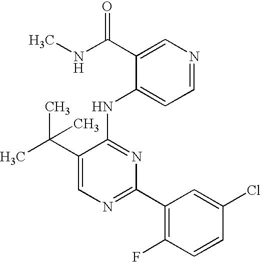 |

Further TGF-β inhibitors for use in the methods of the present invention are represented by (5):

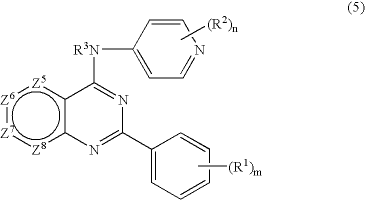

or the pharmaceutically acceptable salts thereof; wherein:

each of Z⁵, Z⁶, Z⁷ and Z⁸ is N or CH and wherein one or two Z⁵, Z⁶, Z⁷ and Z⁸ are N and wherein two adjacent Z positions cannot be N;

m and n are each independently 0-3;

R¹ is halo, alkyl, alkoxy or alkyl halide and wherein two adjacent R¹ groups may be joined to form an aliphatic heterocyclic ring of 5-6 members;

R² is a noninterfering substituent; and

R³ is H or CH₃.

In a preferred embodiment, the small organic molecules herein are derivatives of quinazoline and related compounds containing mandatory substituents at positions corresponding to the 2- and 4-positions of quinazoline. Preferably, the compounds of the invention include a pteridine or pyrido pyrimidine nucleus. Pteridine and 8-pyrido pyrimidine nuclei are preferred. Thus, in one embodiment Z⁵ and Z⁸ are N, and Z⁶ and Z⁷ are CH. However in all cases, at least one of each of Z⁵ -Z⁸ must be N. Preferred embodiments for R¹ are halo, preferably F, Cl, I or Br, most preferably Cl or F; NR₂; OH; or CF₃.

The position that corresponds to the 2-position of the quinazoline contains a mandatory phenyl substituent.

The position that corresponds to the 4-position of the quinazoline contains a mandatory —NR₃ -4′-pridyl substituent that may optionally contain 0-4 non-interfering substituents, namely (R²)ₙ, wherein n is 0-4 preferably, the pyridyl group is unsubstituted, i.e., n is 0. When substituted, the pyridyl moiety is preferably substituted with an alkyl group such as methyl or ethyl, or a halo group preferably bromo or iodo each of which are preferably substituted at the ortho position relative to the pyridyl's linkage to the quinazoline derivative nucleus. In another embodiment, n is 1, and R³ is methyl, preferably, at the 1′ or 2′ position.

The R¹ substituent(s) preferably include minimally bulky groups such as halo, lower alkyl, lower alkoxy, and lower alkyl halide groups. Preferably such groups include one or more halo, such as Cl, F, Br, and I which may be the same or different if more than two halo groups are present; alkyl halide containing 1-3 halides, preferably methyl halide and even more preferably trifluoro methyl; OH; R which is a lower alkyl, preferably C1-6, more preferably C1-3 alkyl, and even more preferably, methyl, ethyl, propyl or isopropyl, most preferably methyl; OR were R is defined as above and OR is preferably methoxy, ethoxy, isopropoxy, methyl phenyloxy. Two adjacent R groups may join to make an aliphatic or hetero aliphatic ring fused to the 2-phenyl. Preferably, if a fused ring is present it has 5 or 6 members, preferably 5 members and contains 1 or more heteroatoms such as N, S or O, and preferably O. Preferably, the fused ring is 1, 3 dioxolane fused to phenyl at the 4 and 5 position of the phenyl ring.

The R1 group or groups that are bound to the 2-phenyl group may be bound at any available position of the phenyl ring. Preferably the R¹ group is bound at the position meta relative to the phenyl's attachment point on the quinazoline derivative nucleus. Also, in a preferred embodiment when phenyl is substituted with two groups, the groups are bound at the ortho and meta positions relative to the phenyl's attachment to the quinazoline derivative, more preferably at non-adjacent ortho and meta positions. Other embodiments include such groups at the ortho or para positions. A phenyl substituted at both meta positions or adjacent ortho and meta positions are contemplated if two groups are present. Alternatively, two groups may form a fused ring preferably attached at the meta and para positions relative to the phenyl's attachment to the quinazoline derivative. Also it is contemplated the phenyl is unsubstituted.

For compounds containing pyridopyrimidine as the nucleus, when the 6- or 7-isomers thereof are present, i.e. the nitrogen is in position 6 or 7 of pyridopyrimidine, the phenyl preferably is unsubstituted, or preferably contains one halo substituent, preferably chlorine, and preferably attached at the meta position relative to the phenyl's attachment to the pyridopyrimidine moiety.

Preferably, the phenyl is substituted, preferably with halo, more preferably one or two halos, and even more preferably chloro at the meta or para positions relative to the phenyl's attachment to the pyridopyrimidine moiety or dichloro at both meta positions; or more preferably substituted with fluoro, preferably difluoro, preferably at the ortho and meta positions relative to the phenyl's attachment to the pyridopyrimidine moiety; or more preferably bromo, preferably at the meta position relative to the phenyl's attachment to the pyridopyrimidine moiety; or more preferably iodo, preferably at the meta position relative to the phenyl's attachment to the pyridopyrimidine moiety.

In another preferred embodiment of compounds containing 8-pyridopyrimidine, the phenyl group is substituted with two or more different halo substituents, preferably disubstituted, and preferably contains fluoro and chloro, and more preferably disubstituted at the non-adjacent ortho and meta positions relative to the phenyl's attachment to the pyridopyrimidine moiety, more preferably where fluoro, is at the ortho position and chloro is at the meta position relative to the phenyl's attachment to the pyridopyrimidine moiety; or preferably is disubstituted with fluoro and bromo, preferably at the non-adjacent ortho and meta positions relative to the phenyl's attachment to the pyridopyrimidine moiety, more preferably where fluoro is at the ortho position and bromo is at the meta position relative to the phenyl's attachment to the pyridopyrimidine moiety.

In another preferred embodiment in compounds containing 8-pyridopyrimidine, the phenyl group is substituted, preferably at one or two positions, and is preferably substituted with alkoxy or arylaryloxy, preferably methoxy, ethoxy isopropoxy, or benzoxy, and preferably at the ortho or meta position relative to the phenyl's attachment to the pyridopyrimidine moiety. In another embodiment in compounds containing 8-pyridopyrimidine, the phenyl is preferably substituted with alkyl, preferably methyl, and preferably at the meta position relative to the phenyl's attachment to the pyridopyrimidine moiety.

In another preferred embodiment in compounds containing 8-pyridopyrimidine, two or more substituents may join to form a fused ring. Preferably the fused ring is a dioxolane ring, more preferably a 1,3-dioxolane ring, fused to the phenyl ring at the meta and para positions relative to the phenyl's attachment to the pyridopyrimidine moiety.

In another preferred embodiment of compounds containing 8-pyrpyriyrimidine, the phenyl group is substituted with two or more different substituents, preferably disubstituted, and preferably chloro and methoxy, and preferably disubstituted at the non-adjacent ortho and meta positions relative to the phenyl's attachment to the pyridopyrimidine moiety, more preferably where methoxy is at the ortho position and chloro is at the meta position relative to the phenyl's attachment to the pyridopyrimidine moiety; or preferably is disubstituted with fluoro and methoxy, preferably at the adjacent ortho and meta positions relative to the phenyl's attachment to the pyridopyrimidine moiety, more preferably where fluoro is at the ortho position and methoxy is at the meta position relative to the phenyl's attachment to the pyridopyrimidine moiety.

In addition, in compounds containing the pteridine nucleus, the phenyl group preferably contains at least one halo substituent at the ortho, meta or para positions relative to the phenyl's attachment to the pteridine moiety. In a more preferred embodiment, the phenyl group contains one chloro group at the ortho or meta positions relative to the phenyl's attachment to the pteridine moiety; one fluoro group at the ortho, meta or para positions relative to the phenyl's attachment to the pteridine moiety; or one bromo or iodo at the meta position relative to the pheniyl's attachment to the pteridine moiety. In another preferred embodiment, the phenyl group contains two halo groups, preferably difluoro, preferably disubstituted at the non-adjacent ortho and meta positions relative to the phenyl's attachment to the pteridine moiety; preferably dichloro, preferably disubstituted at the adjacent ortho and meta positions relative to the phenyl's attachment to the pteridine moiety; preferably fluoro and chloro, preferably disubstituted at the adjacent or non-adjacent ortho, and meta positions relative to the phenyl's attachment to the pteridine moiety, preferably where the fluoro is at the ortho position, and the chloro is at either meta position, and even more preferably where the chloro is at the non-adjacent meta position; or preferably fluoro and bromo preferably substituted at the nonadjacent ortho and meta positions relative to the phenyl's attachment to the pteridine moiety, preferably where the fluoro is at the ortho position, and the bromo is at the non-adjacent meta position.

In another preferred embodiment in compounds containing pteridine, the phenyl group is substituted, preferably at one or more positions, preferably one position, and more preferably with alkoxy, even more preferably with methoxy, and preferably at the ortho or meta position relative to the phenyl's attachment to the pteridine moiety. In another embodiment in compounds containing pteridine, the phenyl is preferably substituted with haloalkyl, preferably trifluoromethyl, and preferably at the meta position relative to the phenyl's attachment to the pteridine moiety.

In another preferred embodiment of compounds containing pteridine, the phenyl group is substituted with two or more different substituents, preferably two substituents, and preferably disubstituted with halo and haloalkyl, more preferably fluoro and trifluoromethyl, and preferably disubstituted at the non-adjacent ortho and meta positions relative to the phenyl's attachment to the pteridine moiety, more preferably where fluoro is at the ortho position and trifluoromethyl is at the meta position relative to the phenyl's attachment to the pteridine moiety.

According to the definition above, R2 is a noninterfering substituent. preferably, R2 is independently H, halo, alkyl, alkenyl, alkynyl, acyl 9or hetero-forms thereof. More preferably R2 is lower alkyl (1-3C), halo such as Br, I, Cl or F. Even more preferably, R2 is methyl, ethyl, bromo, iodo or CONHR. Most preferably, R2 is H.

The following provisos apply to the molecules of formula (5):

when Z⁵-Z⁷ are CH and Z⁸ is N, R¹ is not 2-fluoro, 2-chloro or the phenyl is not unsubstituted;

when Z⁵ and Z⁸ are N and Z⁶ and Z⁷ are CH, the phenyl is not unsubstituted; and

when Z⁵ is N and Z⁶-Z⁸ are CH, the phenyl is not unsubstituted.

Representative compound of formula (5) are listed in the following Table 5.

TABLE 5

| COM- | |
|---|---|
| POUND # | STRUCTURE |
| 253 | 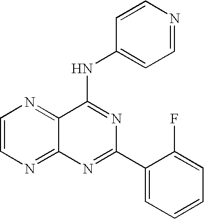 |
| 254 | 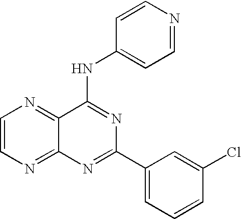 |
| 255 | 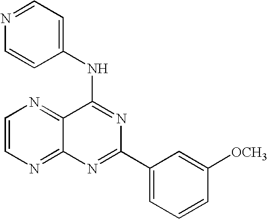 |
| 256 | 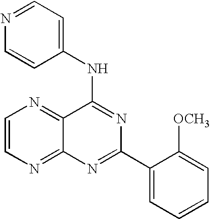 |
| 257 | 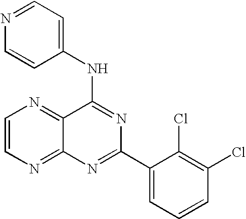 |
| 258 | 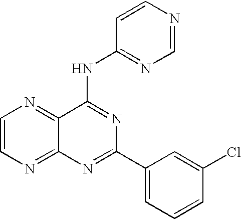 |
| 259 | 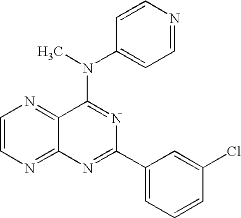 |
| 260 | 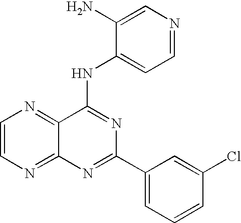 |
| 261 | 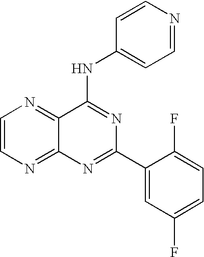 |
| 262 | 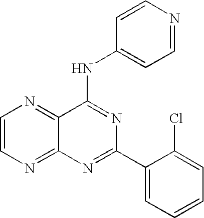 |
| 263 | 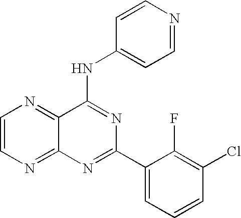 |
| 264 | 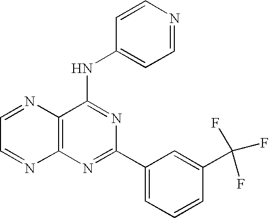 |
| 265 | 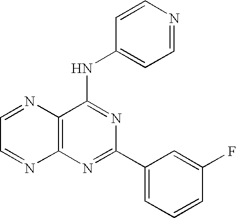 |
| 266 | 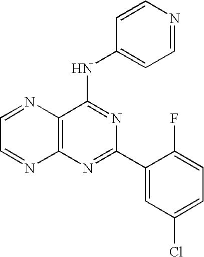 |
| 267 | 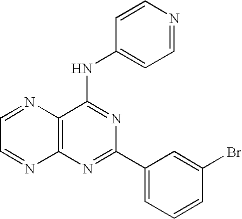 |
| 268 | 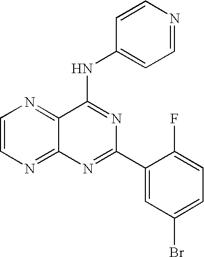 |
| 269 | 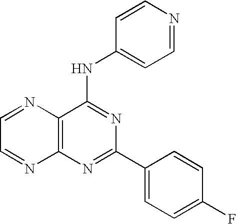 |
| 270 | 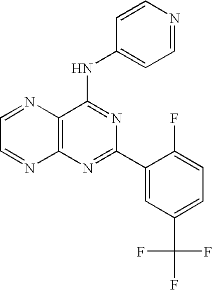 |
| 271 | 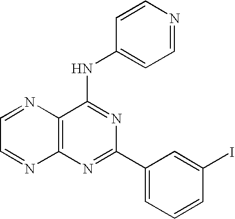 |
| 272 | 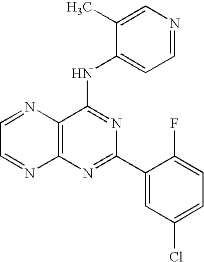 |
| 273 | 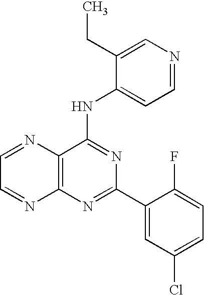 |
| 274 | 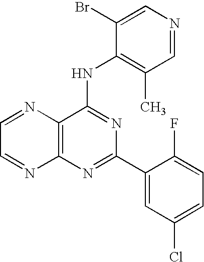 |
| 275 | 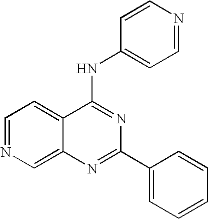 |
| 276 | 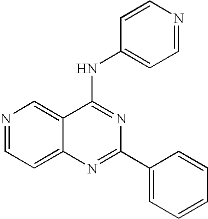 |
| 277 | 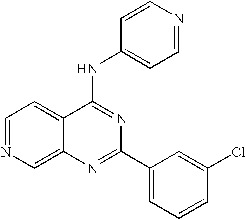 |
| 278 | 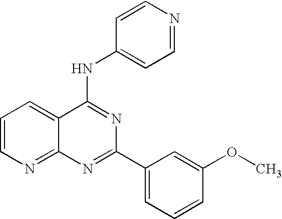 |
| 279 | 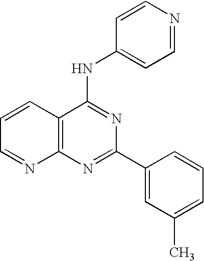 |
| 280 | 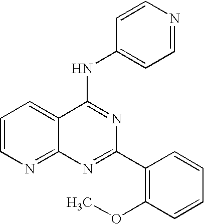 |
| 281 | 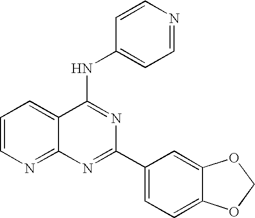 |
| 282 | 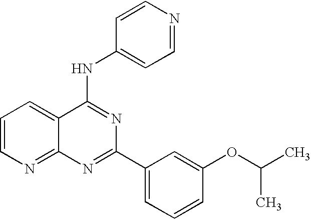 |
| 283 | 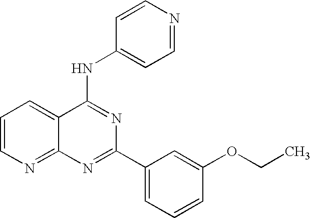 |
| 284 | 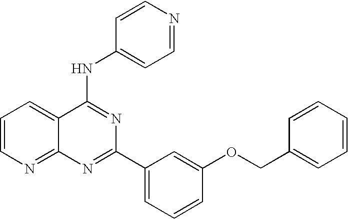 |
| 285 | 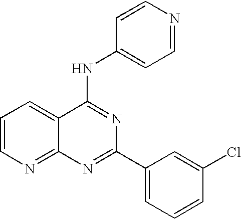 |
| 286 | 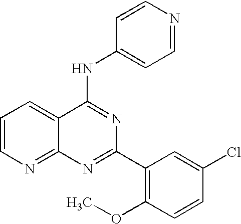 |
| 287 | 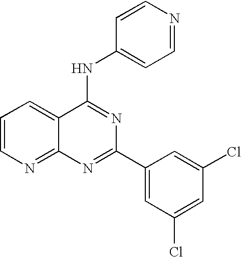 |
| 288 | 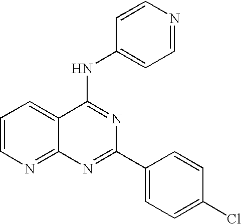 |
| 289 | 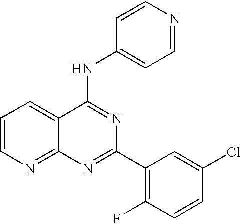 |
| 290 | 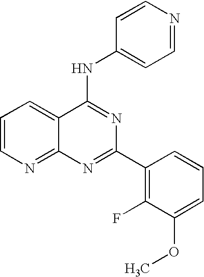 |
| 291 | 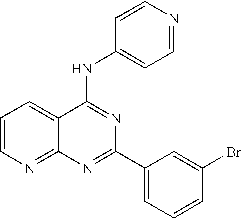 |
| 292 | 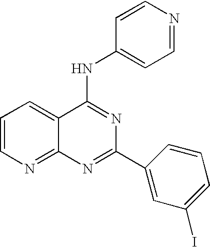 |
| 293 | 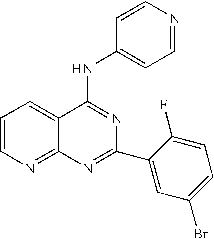 |
| 294 | 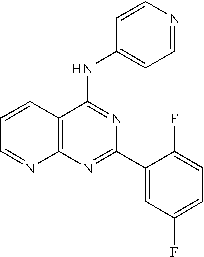 |
| 295 | 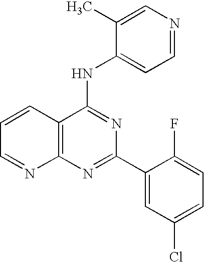 |
| 296 | 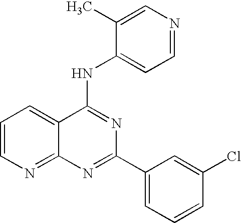 |

The TGF-β inhibitors herein can also be supplied in the form of a “prodrug” which is designed to release the compounds when administered to a subject. Prodrug form designs are well known in the art, and depend on the substituents contained in the compound. For example, a substituent containing sulfhydryl could be coupled to a carrier which renders the compound biologically inactive until removed by endogenous enzymes or, for example, by enzymes targeted to a particular receptor or location in the subject.

In the event that any of the substituents of the foregoing compounds contain chiral centers, as some, indeed, do, the compounds include all stereoisomeric forms thereof, both as isolated stereoisomers and mixtures of these stereoisomeric forms.

Small organic molecules other than quinazoline derivatives can be synthesized by well known methods of organic chemistry as described in standard textbooks.

Methods for the preparation of the compounds of formula (1) are also disclosed, for example, in PCT Publication No. WO 00/12497, published Mar. 9, 2003, the entire disclosure of which is hereby expressly incorporated by reference. Compounds of formula (2) and formula (3), along with methods for their preparation, are disclosed in PCT Publication Nos. WO 02/40468, published May 23, 2002, and WO 00/61576, published Oct. 19, 2000, the entire disclosures of which are hereby expressly incorporated by reference.

Compounds of formula (4) or (5) can be synthesized by methods well known in the art that will be readily apparent for those skilled in the art. For example, Compounds of formula (4) along with methods for their preparation, are disclosed in PCT Application No. PCT/US03/28590, the entire disclosure of which is hereby expressly incorporated by reference. Compounds of formula (5) along with methods for their preparation, are disclosed in U.S. Application No. 60/507,910, the entire disclosure of which is hereby expressly incorporated by reference. In addition, representative compounds within the scope of the invention are further described in U.S. Application No. 60/458,982, the entire disclosure of which is hereby expressly incorporated by reference.

### D. Methods of Treatment

The manner of administration and formulation of the compounds useful in the invention and their related compounds will depend on the nature and severity of the condition, the particular subject to be treated, and the judgment of the practitioner. The particular formulation will also depend on the mode of administration.

Thus, the small molecule compounds of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may further be administered by injection, including intravenous, intramuscular, subcutaneous, intraarticular or intraperitoneal injection. Typical formulations for such use are liquid formulations in isotonic vehicles such as Hank's solution or Ringer's solution.

Alternative formulations include aerosol inhalants, nasal sprays, liposomal formulations, slow-release formulations, and the like, as are known in the art.

Any suitable formulation may be used.

A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

As implicated above, although the compounds of the invention may be used in humans, they are also available for veterinary use in treating non-human mammalian subjects.

Further details of the invention will be apparent from the following non-limiting examples.

### EXAMPLE 1

### Study of the Effect of a TGFβ-R1 Inhibitor on Obesity in db/db Mice

Study Design

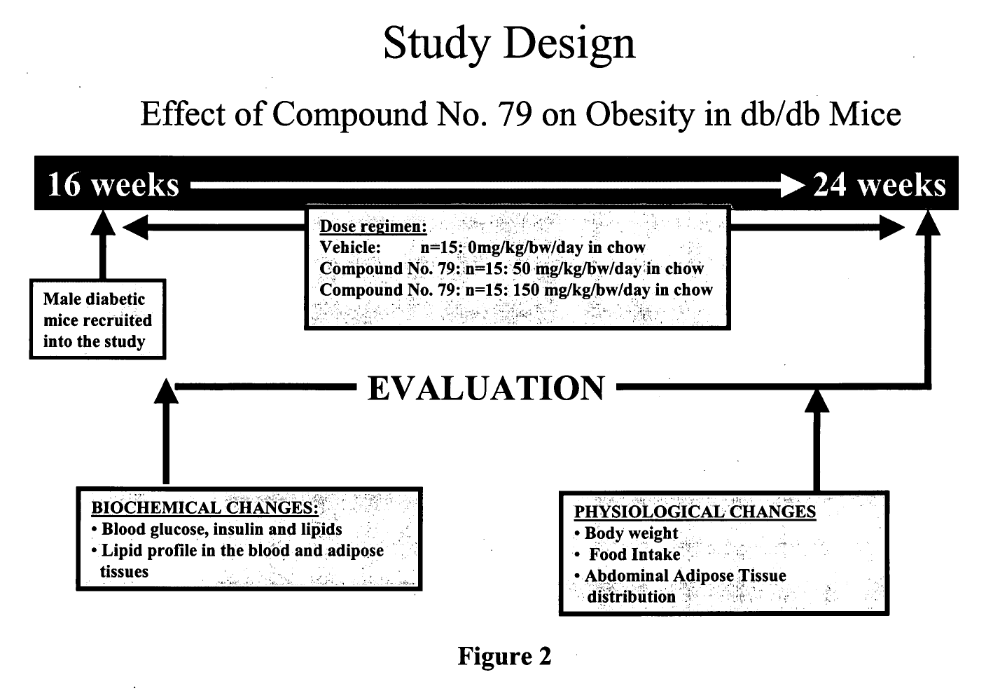

As shown in FIG. 2, 45 16-week-old male diabetic db/db mice were recruited into the study, and divided into three groups, each containing 15 animals. Group 1 (control) was administered vehicle (chow) alone; Group 2 received 50 mg/kg/body weight/day of a representative TGF-β inhibitor (Compound No. 79) in chow; and Group 3 received 150 mg/kg/body weight/day of Compound No. 79 in chow. Physiological and biochemical changes, for example, changes in body weight, food intake, abdominal fat distribution and blood glucose, were evaluated at 24 weeks of age.

Results

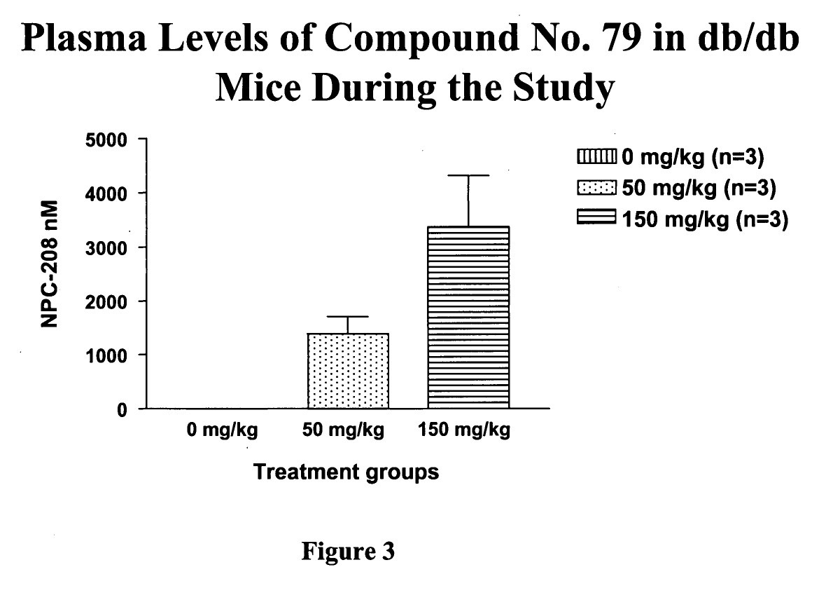

FIG. 3 shows the plasma levels of a representative TGF-β inhibitor (Compound No. 79) in db/db mice during the study.

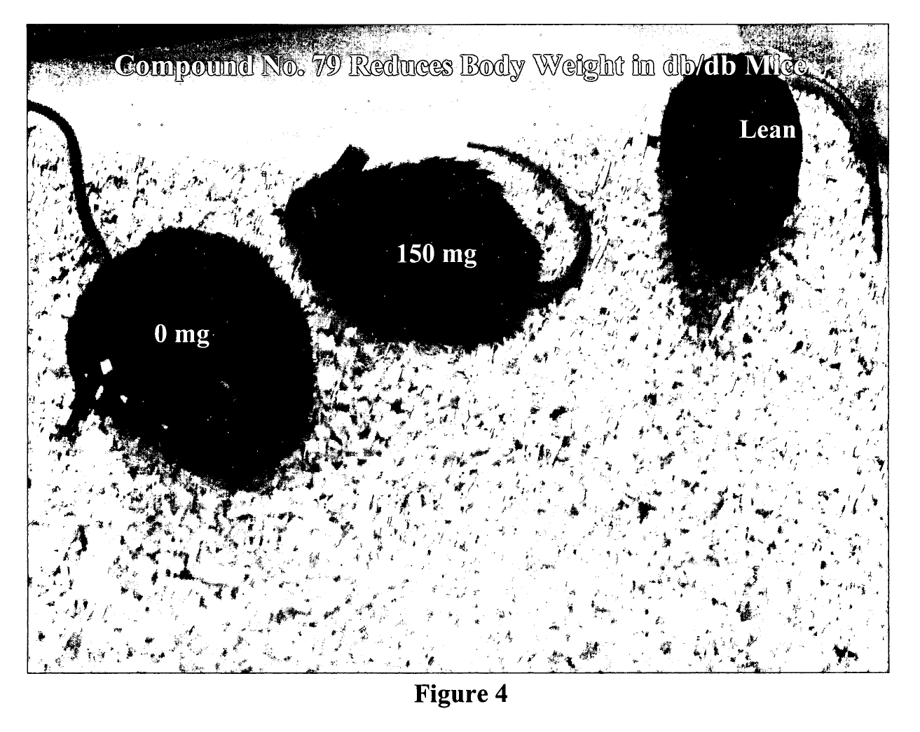

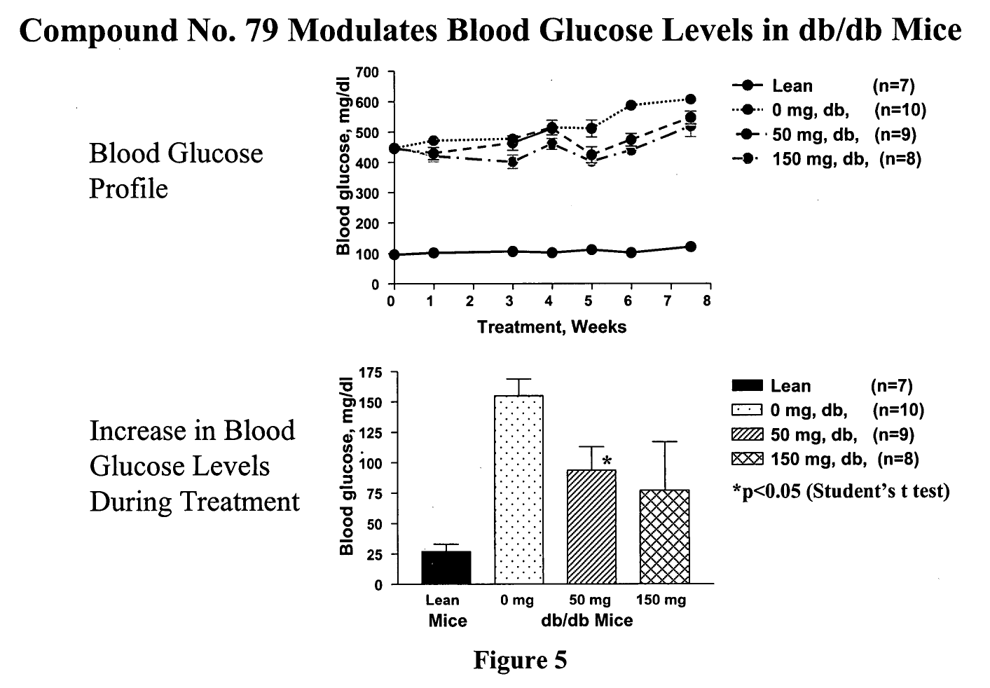

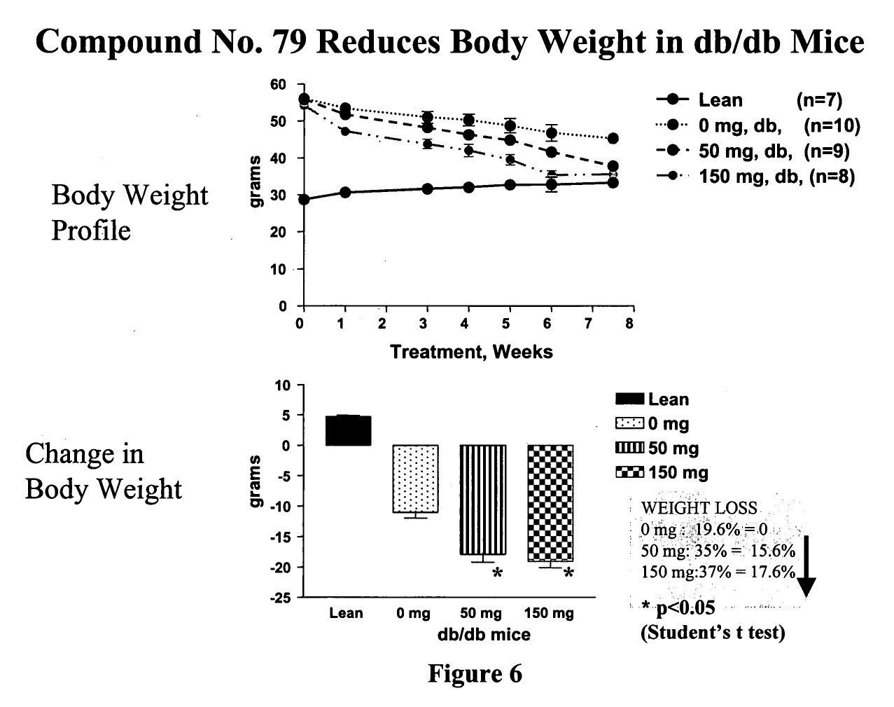

As shown in FIG. 4, administration of 150 mg/kg/body-weight/day of Compound No. 79 significantly reduced the body weight of db/db obese mice. The results of administrating 50 mg and 150 mg/kg/body weight/day of Compound No. 79 same is shown quantitatively in FIG. 6. Administration of 50 mg and 150 mg/kg/body-weight/day of Compound No. 79 reduced the body weight of db/db obese mice in a statistically significant manner. [0134] FIG. 5 shows the blood glucose profile and the increase in blood glucose levels during the course of treatment in lean mice and in Groups 1-3 of db/db mice treated as described above. As seen in FIG. 5, the blood glucose level increases in all categories of mice, namely lean mice and mice in Groups 1-3. The increase in blood glucose level is highest in control mice that received chow alone; i.e., 0 mg/kg/body-weight/day of Compound No. 79. The rise in blood glucose levels in mice that received either 50 or 150 mg/kg/body weight/day of Compound No. 79 was significantly less, indicating that Compound 79 effectively modulates blood glucose levels.

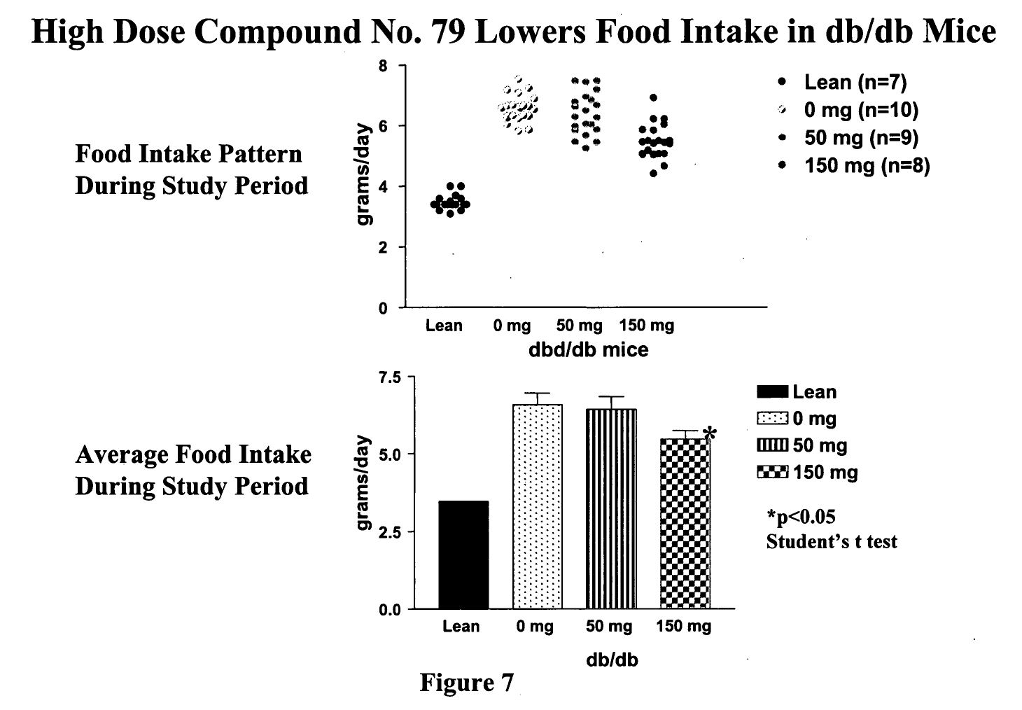

FIG. 7 shows the food intake pattern and the average food intake during the study period. As seen in FIG. 7, the food intake of mice that received 150 mg/kg/body-weight/day of Compound No. 79 was significantly less, indicating that Compound 79 lowers food intake of db/db mice in a statistically significant manner.

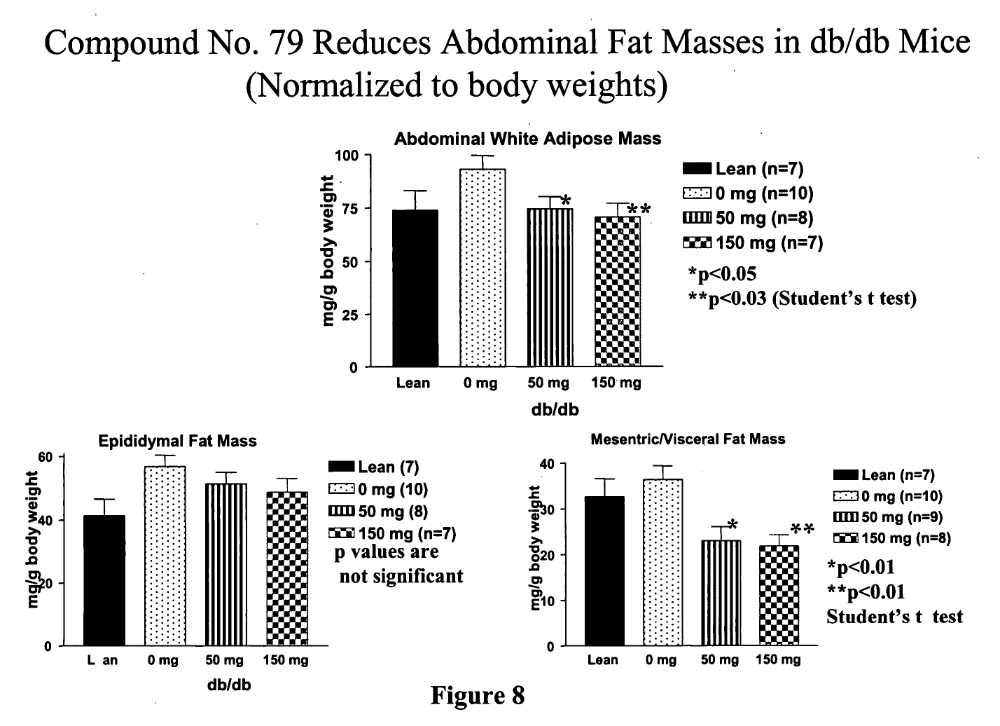

FIG. 8 shows the reduction of abdominal fat masses in db/db mice, normalized to body weights, as a result of administration of Compound No. 79. As can be seen from FIG. 8, Compound No. 79 is effective, at doses of both 50 and 150 mg/kg/body-weight/day, in reducing abdominal fat mass in a statistically significant manner.

Conclusions

In this set of experiments, the representative TGF-β inhibitor tested at the highest dose (150 mg/kg/body weight/day) reduced food intake and body weight in a statistically significant manner. The low dose (50 mg/kg/body weight/day) also reduced body weight gain in a statistically significant manner with marginal effect on food intake. In addition, the TGF-β inhibitor reduced abdominal fat masses in a statistically significant manner both in low and high doses. Similarly, both high and low doses of the TGF-β inhibitor tested modulated blood glucose levels, controlling the rise seen in its absence.

In conclusion, the data of these experiments show that the representative TGF-β inhibitor (Compound No. 79) significantly restricts food intake and causes loss of body weight in a well-established animal model of obesity. As a result, the test compound and other TGF-β inhibitors are promising drug candidates for the prevention and treatment of obesity and related pathologic conditions, including type 2 diabetes.

The results of this study can be further validated by repeating essentially the same experiment with lower doses of a TGF-β inhibitor, and on a larger number of db/db mice for 8 weeks, with special emphasis on the assessment of muscle growth. Other follow-up studies might include in vivo adipogenesis studies on db/db mice of 4 weeks old, in vitro adipogenesis studies on 3T3L1 cells, in vivo gene microarray studies, and glucose tolerane studies. The results can be further be validated in one or more further rodent models of obesity, such as those discussed above.

In general, there are three basic mechanisms that can be used to classify drug treatments for obesity: (1) drugs that reduce food intake, (2) drugs that affect metabolism, and (3) drugs that increase energy expenditure. Any agent that can reduce food intake holds promise for the treatment of obesity. The mechanism resulting in the reduction of food intake can be noradrenergic, serotonergic, dopaminergic and histaminergic. TGF-β inhibition has a beneficial effect on appetite suppression. Without being bound by any particular theory, this appetite suppression seems to work via the noradrenergic mechanism. As discussed before, the data on the TGF-β inhibitor tested suggest that it lowers body fat mass by reducing food intake. Accordingly, the TGF-β inhibitors of the present invention find utility as appetite suppressive drugs.

All references cited throughout the specification are expressly incorporated herein by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, and the like. All such modifications are within the scope of the claims appended hereto.

What is claimed is:

1. A method for the treatment of obesity or a pathologic condition associated with obesity comprising administering to an obese mammalian subject or a mammalian subject at risk of developing obesity a therapeutically effective amount of a compound capable of inhibiting TGF-β signaling through a TGF-β receptor.

2. The method of claim 1 wherein the pathologic condition associated with obesity is selected from the group consisting of, type 2 diabetes, insulin resistance, sexual dysfinction, hypertension, hypercholesterolemia, atherosclerosis, hyperlipoproteinemia, and hypertriglyceridemia.

3. The method of claim 1 wherein the pathologic condition associated with obesity is type 2 diabetes or a pathologic condition associated with type 2 diabetes.

4. The method of claim 3 wherein the pathologic condition associated with type 2 diabetes is selected from the group consisting of diabetic retinopathy, diabetic neuropathy, hypertension, atherosclerosis, diabetic ulcers, and damage caused to blood vessels, nerves and other internal structures by elevated blood sugar levels.

5. The method of claim 1 wherein the mammalian subject is human.

6. The method of claim 5 wherein the human subject is at risk of developing obesity.

7. The method of claim 5 wherein the human subject is obese.

8. The method of claim 5 wherein the human subject has been diagnosed with type 2 diabetes.

9. The method of claim 1 wherein the TGF-β receptor is a TGFβ-R1 kinase.

10. The method of claim 9 wherein said compound is capable of binding to the TGFβ-R1 kinase.

11. The method of claim 10 wherein the compound is a non-peptide small molecule.

12. The method of claim 11 wherein the compound is a small organic molecule.

13. The method of claim 12 wherein the small organic molecule is a compound of formula (1): 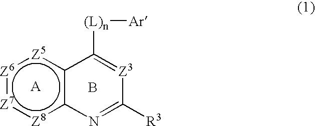
or the pharmaceutically acceptable salts or prodrug forms thereof; wherein:
R³ is a noninterfering substituent;
each Z is CR² or N, wherein no more than two Z positions in ring A are N, and wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

14. The method of claim 13 wherein the compound is a quinazoline derivative.

15. The method of claim 13 wherein wherein Z³ is N; and Z⁵-Z⁸ are CR².

16. The method of claim 13 wherein Z³ is N; and at least one of Z⁵-Z⁸ is nitrogen.

17. The method of claim 13 wherein R³ is an optionally substituted phenyl moiety.

18. The method of claim 17 wherein R³ is selected from the group consisting of 2-, 4-, 5-, 2,4- and 2,5-substituted phenyl moieties.

19. The method of claim 18 wherein at least one substituent of the phenyl moiety is an alkyl(1-6C), or halo.

20. The method of claim 12, wherein the small organic molecule is a compound of formula (2): 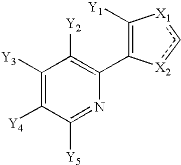
or the pharmaceutically acceptable salts or prodrug forms thereof; wherein:
Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ—Ph, —S—(CH₂)ₘ—Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and
Y₂, Y₃, Y₄, and Y₅ independently represent hydrogen, alkyl(1-6C), alkoxy(I-6 C), haloalkyl(1-6 C), halo, NH₂, NH-alkyl(1-6C), or NH(CH₂)ₙ—Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙPh, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ—Ph wherein n is 0-3; and
one of X₁ and X₂ is N and the other is NR₆, wherein R₆ is hydrogen or alkyl(1-6 C).

21. The method of claim 12 wherein said small organic molecule is a compound of formula (3): 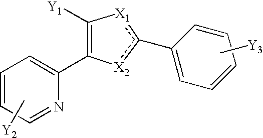
or the pharmaceutically acceptable salts or prodrug forms thereof; wherein:
Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)—Ph, —S—(CH₂)ₙ—Ph, cyano, phenyl, and CO₂R, wherein R is hydrogen or alkyl(1-6 C), and n is 0, 1, 2, or 3; or Y, represents phenyl fused with an aromatic or non-aromatic cyclic ring of 5-7 members wherein said cyclic ring optionally contains up to two heteroatoms, independently selected from N, O, and S;
Y₂ is H, NH(CH₂)ₙ—Ph or NH-alkyl(1-6 C), wherein n is 0, 1, 2, or 3;
Y₃ is CO₂H, CONH₂, CN, NO₂, alkylthio(1-6 C), —SO₂-alkyl(C1-6), alkoxy(C1-6), SONH₂, CONHOH, NH₂, CHO, CH₂NH₂, or CO₂R, wherein R is hydrogen or alkyl(1-6 C);
one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

22. The method of claim 12 wherein said small organic molecule is a compound of formula (4): 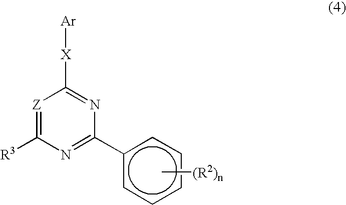
or the pharmaceutically acceptable salts or prodrug forms thereof; wherein:
Ar represents an optionally substituted aromatic or optionally substituted heteroaromatic moiety containing 5-12 ring members wherein said heteroaromatic moiety contains one or more O, S, and/or N with a proviso that the optionally substituted Ar is not 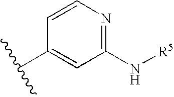
wherein R⁵ is H, alkyl (1-6C), alkenyl (2-6C), alkynyl (2-6C), an aromatic or heteroaromatic moiety containing 5-11 ring members;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

23. The method of claim 12 wherein said small organic molecule is a compound of formula (5): 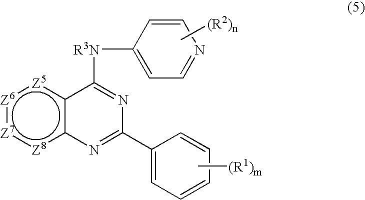
or the pharmaceutically acceptable salts or prodrug forms thereof; wherein:
each of Z⁵, Z⁶, Z⁷ and Z⁸ is N or CH and wherein one or two Z⁵, Z⁶, Z⁷ and Z⁸ are N and wherein two adjacent Z positions cannot be N;
m and n are each independently 0-3;
R¹ is halo, alkyl, alkoxy or alkyl halide and wherein two adjacent R¹ groups may be joined to form an aliphatic heterocyclic ring of 5-6 members;
R² is a noninterfering substituent; and
R³ is H or CH₃.

24. A method for the treatment of type 2 diabetes comprising administering to a mammalian subject diagnosed with or at risk of developing type 2 diabetes a therapeutically effective amount of a compound capable of inhibiting TGF-β signaling through a TGF-β receptor.

25. A method for appetite suppression comprising administering to a mammalian subject in need an effective amount of a compound capable of inhibiting TGF-β signaling through a TGF-β receptor.

26. A method for limiting food intake in a mammalian subject comprising administering to said subject an effective amount of a compound capable of inhibiting TGF-β signaling through a TGF-β receptor.
